(19) United States
(12) Patent Application Publication
(10) Pub. No.: US 2003/0235595 A1
(43) Pub. Date: Dec. 25, 2003

(54) Oil-containing, orally administrable pharmaceutical composition for improved delivery of a therapeutic agent

(76) Inventors: Feng-Jing Chen, Salt Lake City, UT (US); Mahesh V. Patel, Salt Lake City, UT (US)

Correspondence Address: REED & EBERLE LLP, 800 MENLO AVENUE, SUITE 210, MENLO PARK, CA, 94025, US

(21) Appl. No.: 10/397,969

(22) Filed: Mar. 25, 2003

Related U.S. Application Data
(63) Continuation-in-part of application No. 09/877,541, filed on Jun. 8, 2001.
Application No. 09/877,541 is a continuation-in-part of application No. 09/345,615, filed on Jun. 30, 1999, now Pat. No. 6,267,985.
Application No. 09/877,541 is a continuation-in-part of application No. 09/751,968, filed on Dec. 29, 2000, now Pat. No. 6,458,383.
Application No. 09/751,968 is a continuation-in-part of application No. 09/375,636, filed on Aug. 17, 1999, now Pat. No. 6,309,663.

(51) Int. Cl. A61K 9/00; A61K 31/192

(52) U.S. Cl. 424/400; 514/571

(57) ABSTRACT

The present invention relates to oral pharmaceutical compositions and methods for improved delivery of therapeutic agents, e.g., lipid-regulating agents. Compositions of the present invention include a carrier, where the carrier contains a combination of a triglyceride and at least two surfactants, at least one of which is hydrophilic. Upon dilution with an aqueous medium, the composition forms a clear, aqueous dispersion. The invention also pertains to methods for treating lipid disorders such as hypercholesterolemia, hypertriglyceridemia, and mixed dyslipidemia by oral administration of the compositions provided.

### CROSS-REFERENCE TO RELATED APPLICATIONS

This application is a continuation-in-part of U.S. patent application Ser. No. 09/877,541, filed Jun. 8, 2001, which is a continuation-in-part of U.S. patent application Ser. No. 09/345,615, filed Jun. 30, 1999, issued on Jul. 31, 2001 as U.S. Pat. No. 6,267,985, and of U.S. application Ser. No. 09/751,968, filed Dec. 29, 2000, issued on Oct. 1, 2002 as U.S. Pat. No. 6,458,383, which is a continuation-in-part of U.S. application Ser. No. 09/375,636, filed Aug. 17, 1999, issued Oct. 30, 2001 as U.S. Pat. No. 6,309,663, the disclosures of which are hereby incorporated by reference.

### TECHNICAL FIELD

The present invention relates to pharmaceutical compositions for oral administration of a therapeutic agent, and more particularly relates to oil-containing pharmaceutical compositions for oral administration of a hydrophobic therapeutic agent, e.g., a lipid-regulating agent. The invention additionally pertains to a method for treating physiological disorders, conditions, and diseases, e.g., lipid disorders such as hypercholesterolemia, hypertriglyceridemia, and mixed dyslipidemia. The invention has utility in the fields of pharmaceutical formulation, pharmacology, and medicine.

### BACKGROUND

A wide variety of therapeutic agents are conventionally formulated in oil/water emulsion systems. These conventional emulsions take advantage of the increased solubility of many therapeutic agents in oils, i.e., triglycerides. Thus, one conventional approach is to solubilize a therapeutic agent in a bioacceptable triglyceride solvent, such as a digestible vegetable oil, and disperse this oil phase in an aqueous medium. The dispersion may be stabilized by emulsifying agents and provided in emulsion form. Alternatively, the therapeutic agent can be provided in a water-free formulation, with an aqueous dispersion being formed in vivo in the gastrointestinal environment. The properties of these oil-based formulations are determined by such factors as the size of the triglyceride/therapeutic agent colloidal particles and the presence or absence of surfactant additives.

In simplest form, a triglyceride-containing formulation suitable for delivering therapeutic agents through an aqueous environment is an oil-in-water emulsion. Such emulsions contain the therapeutic agent solubilized in an oil phase that is dispersed in an aqueous environment with the aid of a surfactant. The surfactant may be present in the oil-based formulation itself, or may be a compound provided in the gastrointestinal system, such as bile salts, which are known to be in vivo emulsifying agents. The colloidal oil particle sizes are relatively large, ranging from several hundred nanometers to several microns in diameter, in a broad particle size distribution. Since the particle sizes are on the order of or greater than the wavelength range of visible light, such emulsions, when prepared in an emulsion dosage form, are visibly “cloudy” or “milky” to the naked eye.

Although conventional triglyceride-based pharmaceutical compositions are useful in solubilizing and delivering some therapeutic agents, such compositions are subject to a number of significant limitations and disadvantages. Emulsions are thermodynamically unstable, and colloidal emulsion particles will spontaneously agglomerate, eventually leading to complete phase separation. The tendency to agglomerate and phase separate presents problems of storage and handling, and increases the likelihood that pharmaceutical emulsions initially properly prepared will be in a less optimal, less effective, and poorly-characterized state upon ultimate administration to a patient. Uncharacterized degradation is particularly disadvantageous, since increased particle size slows the rate of transport of the colloidal particle and digestion of the oil component, and hence the rate and extent of absorption of the therapeutic agent. These problems lead to poorly characterized and potentially harmful changes in the effective dosage received by the patient. Moreover, changes in colloidal emulsion particle size are also believed to render absorption more sensitive to and dependent upon conditions in the gastrointestinal tract, such as pH, enzyme activity, bile components, and stomach contents. Such uncertainty in the rate and extent of ultimate absorption of the therapeutic agent severely compromises the medical professional's ability to safely administer therapeutically effective dosages. In addition, when such compositions are administered parenterally, the presence of large particles can block blood capillaries, further compromising patient safety.

A further disadvantage of conventional triglyceride-containing compositions is the dependence of therapeutic agent absorption on the rate and extent of lipolysis. Although colloidal emulsion particles can transport therapeutic agents through the aqueous environment of the gastrointestinal tract, ultimately the triglyceride must be digested and the therapeutic agent must be released in order to be absorbed through the intestinal mucosa. The triglyceride carrier is emulsified by bile salts and hydrolyzed, primarily by pancreatic lipase. The rate and extent of lipolysis, however, are dependent upon several factors that are difficult to adequately control. For example, the amount and rate of bile salt secretion affect the lipolysis of the triglycerides, and the bile salt secretion can vary with stomach contents, with metabolic abnormalities, and with functional changes of the liver, bile ducts, gall bladder, and intestine. Lipase availability in patients with decreased pancreatic secretory function, such as cystic fibrosis or chronic pancreatitis, may be undesirably low, resulting in a slow and incomplete triglyceride lipolysis. The activity of lipase is pH dependent, with deactivation occurring at about pH 3, so that the lipolysis rate will vary with stomach contents, and may be insufficient in patients with gastric acid hyper-secretion. Moreover, certain surfactants commonly used in the preparation of pharmaceutical emulsions, such as polyethoxylated castor oils, may themselves act as inhibitors of lipolysis. Although recent work suggests that certain surfactant combinations, when used in combination with digestible oils in emulsion preparations, can substantially decrease the lipolysis-inhibiting effect of some common pharmaceutical surfactants (see, U.S. Pat. No. 5,645,856), such formulations are still subject to the other disadvantages of pharmaceutical emulsions and triglyceride-based formulations.

Yet another approach is based on formation of “microemulsions.” Like an emulsion, a microemulsion is a liquid dispersion of oil in water, stabilized by surfactants. Conventional microemulsions, however, present several safety and efficiency problems. The amount of triglyceride that can be solubilized in a conventional microemulsion is generally quite small, resulting in a poor loading capacity. In order to solubilize significant amounts of triglycerides, large amounts of hydrophilic surfactant and/or solvents must be used. These high concentrations of hydrophilic surfactant and solvents raise questions of safety, since the levels of hydrophilic surfactant and solvent needed can approach or exceed bioacceptable levels.

Thus, conventional triglyceride-containing formulations suffer from limitations and safety concerns including, for example, instability of the formulation, dependence on lipolysis and poor loading capacity of the therapeutic agent. Triglyceride-containing formulations incorporating a therapeutic agent, in particular a lipid-regulating agent, which do not suffer from these and other limitations and safety concerns are desired.

Effective administration of one particular class of therapeutic agents, lipid-regulating agents, has proven difficult because conventional formulations of such drugs suffer from several disadvantages such as poor bioavailability and a highly variable dissolution profile. To compensate for low bioavailability, the dose is often increased. Dosage increases, however, still do not address the problems associated with highly variable inter- and/or intra-subject bioavailability. Thus, conventional formulations of lipid-regulating agents are frequently required to be taken with meals in order to address poor bioavailability. As a result, however, patient compliance is often low as patients may forget to administer these formulations with meals or decide to skip a dose when the patient is not willing to consume an entire meal. Poor patient compliance, in turn, requires frequent monitoring and dosage adjustments by the treating clinician. In particular, these disadvantages are evidenced with several families of lipid-regulating agents, such as fibrates and statins.

Fenofibrate (2-[4-(4-chlorobenzoyl)phenoxy]-2-methyl-propanoic acid, 1-methylethylester) is a well-known lipid-regulating agent from the fibrate family and is a representative lipid-regulating agent herein. The active metabolite of fenofibrate, fenofibric acid, produces reductions in total cholesterol, low density lipoprotein (LDL), apolipoprotein B, total triglycerides and very low density lipoprotein (VLDL). In addition, treatment with fenofibrate results in increases in high density lipoprotein (HDL).

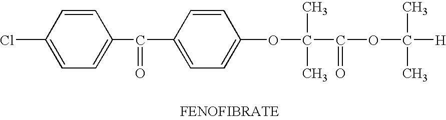

Fenofibrate is hydrophobic in nature (see structure shown above) and is practically insoluble in water. Fenofibrate has been commercially available under the names Lipanthyl, Lipidil® and Lipantil. Although the usual daily dose is as high as 300-400 mg, the product is nonetheless poorly absorbed in the gastrointestinal tract of patients. As a result, it is poorly and variably bioavailable and must be taken with food.

One approach in producing pharmaceutically acceptable fenofibrate formulations involves the use of micronization. U.S. Pat. No. 4,895,726 to Curtet et al., for example, discloses a composition and method of improving the dissolution, and consequently, the bioavailability, of fenofibrate by using a solid surfactant that is co-micronized with fenofibrate. U.S. Pat. No. 5,880,148 to Edgar et al. discloses a combination of a micronized mixture of fenofibrate with a solid surfactant and a vitamin E substance. U.S. Pat. Nos. 4,800,079 to Boyer et al. and 6,074,670 to Stamm et al. also pertain to pharmaceutical formulations containing micronized fenofibrate. In addition, a micronized fenofibrate formulation is commercially available under the name TriCor® from Abbott Laboratories.

The bioavailability of micronized fenofibrate is significantly improved relative to the non-micronized form of the drug, and micronization can, therefore, reduce the required dosage. However, the absorption of fenofibrate from the commercial product, TriCor®, is heavily influenced by the presence of food, necessitating administration with meals. This requirement presents a patient compliance issue for any drug.

Furthermore, the preparation of fenofibrate in the form of crystalline microparticles or that of co-micronizing fenofibrate with a solid surfactant is a time consuming and costly process. An inherent drawback of micronization is that the material obtained must comply with stringent particle size specifications, and the handling and filling of capsules with a micronized powder present challenges with regard to safety and homogeneity of the formulation. Most importantly, micronization of a drug requires complete and consistent dissolution of the drug as a prerequisite for effective absorption and a satisfactory bioavailability profile.

Other approaches for producing fenofibrate formulations have been described. U.S. Pat. No. 5,827,536 to Laruelle, for example, discloses a formulation containing fenofibrate in combination with a solubilizing agent consisting of a non-ionic surfactant, diethylene glycol monoethyl ether (DGME). U.S. Pat. No. 5,545,628 to Deboeck et al. discloses compositions containing fenofibrate and one or more polyglycolyzed glycerides. U.S. Pat. No. 6,096,338 to Lacy et al. discloses a carrier for hydrophobic drugs, e.g., fenofibrate, which contains a digestible oil and a pharmaceutically acceptable surfactant for dispersing the oil in vivo. WO 99/29300 to Mishra discloses a self-emulsifying preconcentrate containing fenofibrate dissolved in a carrier system comprising a hydrophobic component, a surfactant, and a hydrophilic component. Each of these approaches, however, has individual drawbacks. As an example, the fenofibrate formulations described in WO 99/29300 contain a plurality of components that are unsuitable for incorporation into an orally administered pharmaceutical product for human ingestion. These components include Myrj 52, Miglyol 840, and linoleic acid, which have not been incorporated in any oral prescription product approved by the U.S. Food & Drug Administration. Without defined toxicity and irritability levels having been established with regard to oral ingestion of these components by a human at the dosed level, potential lack of safety is a significant drawback of the aforementioned compositions.

Therefore, for more effective management of lipid disorders, there is an ongoing need for improved formulations of fenofibrate and other lipid-regulating agents. In particular, there is a need for formulations that are not dependent on the micronization of the lipid-regulating agent or the co-micronization of the agent with a solid surfactant for effective absorption. Ideal formulations would: provide a superior rate and/or extent of absorption without dependence on lipolysis endogenous bile, bile-related patient disease states, or meal fat contents, and without need for a high drug dose; exhibit chemical and physical stability over extended storage periods; be less costly to manufacture and commercialize than prior formulations of lipid-regulating agents; not require administration with food; and include only those excipients that are pharmaceutically acceptable.

### SUMMARY OF THE INVENTION

The present invention addresses the aforementioned need in the art by providing an improved pharmaceutical composition for the oral administration of a therapeutic agent, preferably a hydrophobic therapeutic agent, e.g., a lipid-regulating agent. The composition exhibits superior bioavailability and absorption without dependence on lipolysis, meal fat contents, or the like, and does not require a high dose of drug, administration with food, or processing via micronization or other potentially limiting and/or costly manufacturing techniques. The invention therefore represents a significant advance in the pharmacotherapeutic management of many physiological disorders, conditions, and diseases, including metabolic disorders, such as may be associated with a metabolic syndrome.

In one embodiment, the composition of the invention is an orally administrable pharmaceutical formulation that comprises a carrier and a therapeutically effective amount of a therapeutic agent, particularly a hydrophobic therapeutic agent, and preferably a lipid-regulating agent, the carrier including a triglyceride and at least two surfactants, at least one of which is hydrophilic, and, optionally, at least one of which is hydrophobic. The triglyceride and surfactants are selected and present in amounts such that upon admixture of the composition with an aqueous medium in an aqueous medium to composition ratio of about 100:1 by weight, either in vitro or in vivo, a clear aqueous dispersion is formed. Generally, this means that the aqueous dispersion exhibits an absorbance of less than about 0.3 at 400 nm. Preferably, the relative amounts of the triglyceride and surfactants should be such that the carrier is capable of containing more triglyceride that can be solubilized relative to an analogous composition containing only a single hydrophilic surfactant.

Following oral administration, the composition provides an increase in the rate of absorption of the therapeutic agent (again, preferably a lipid-regulating agent) relative to the rate of absorption, for a corresponding composition, administered under an identical dosage regimen, containing (a) the therapeutic agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after “dose normalization,” i.e., wherein the relative absorbance values are normalized to account for any difference in the amount of therapeutic agent administered. By “identical dosage regimen,” as used herein, is meant a dosage regimen that is identical not only with respect to drug dose, but also with respect to meal timing and meal content, particularly meal fat content. The increase in the rate of absorption corresponds to and may be determined by the amount of time required to reach maximum plasma concentration of the therapeutic agent or an active metabolite thereof. In a preferred embodiment, then, the increase in the rate of absorption provided by the invention is such that the time to reach maximum plasma concentration of the therapeutic agent or an active metabolite thereof is reduced by at least about 10%. When the therapeutic agent is fenofibrate, a preferred lipid-regulating agent herein, the increase in the rate of absorption corresponds to and may be determined by the amount of time required to reach maximum plasma concentration of fenofibric acid, the active metabolite of fenofibrate.

In addition, following oral administration, the composition provides an increase in the extent of absorption of the therapeutic agent (again, preferably a lipid-regulating agent) relative to the extent of absorption for a corresponding composition, administered under an identical dosage regimen, containing (a) the therapeutic agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization. The increase in the extent of absorption may be determined by the area under the curve (AUC) of the plasma concentration of the therapeutic agent or an active metabolite thereof as a function of time. In a preferred embodiment, the increase in the extent of absorption provided by the invention is such that the AUC of the plasma concentration of the therapeutic agent or an active metabolite thereof is increased by at least about 10%. When the therapeutic agent is fenofibrate, the increase in the extent of absorption corresponds to and may be determined by the AUC of the plasma concentration of fenofibric acid.

In another embodiment, the invention provides a pharmaceutical composition comprising: (a) a carrier comprising a triglyceride and at least two surfactants, at least one of the surfactants being hydrophilic; and (b) a therapeutically effective amount of a therapeutic agent, such as a lipid-regulating agent, wherein the triglyceride and the surfactants are present in amounts that are pharmaceutically acceptable and selected so that upon admixture of the composition with an aqueous medium in an aqueous medium to composition ratio of about 10:1 by weight, a clear aqueous dispersion is provided.

In an additional embodiment, the present invention relates to orally administrable dosage forms comprising the pharmaceutical compositions described herein. The dosage forms may be processed by techniques selected from the group consisting of lyophilization, encapsulation, extruding, compression, melting, molding, spraying, coating, comminution, mixing, homogenization, sonciation, granulation, and combinations thereof. Dosage forms include, but are not limited to, pills, capsules, caplets, tablets, granules, beads, powders, solutions, suspensions, emulsions, syrups, and elixirs. Preferred dosage forms are capsules, e.g., starch, hydroxypropyl methylcellulose, and gelatin capsules. Gelatin capsules, which may be hard or soft, are normally preferred. Generally, although not necessarily, the amount of therapeutic agent in any particular dosage form will be a unit dosage.

In a related embodiment, the invention provides an orally administrable pharmaceutical composition as described above, for the administration of a therapeutic agent, e.g., a hydrophobic therapeutic agent, particularly a lipid-regulating agent such as fenofibrate, wherein the composition does not include any components (e.g., excipients) other than pharmaceutically acceptable components, and no such component is present in a quantity that exceeds a pharmaceutically acceptable level.

The invention also provides a method of treating a lipid disorder in a patient, particularly a human patient, with a lipid-regulating agent, the method involving providing a composition containing a carrier as described herein, providing the lipid-regulating agent, and administering the carrier-containing composition to the patient. The active agent may be administered simultaneously, either in the carrier-containing composition or in a separate composition. Alternatively, the active agent may be administered at a different point in time, i.e., either the agent is administered first followed by administration of the carrier composition, or the carrier composition is administered first followed by administration of the active agent. Generally, the method involves administration of a pharmaceutical composition comprising a carrier and a therapeutically effective amount of a lipid-regulating agent, the carrier including a triglyceride and at least two surfactants, at least one of which is hydrophilic, and, optionally, at least one of which is hydrophobic, wherein the triglyceride and surfactants are selected and present in amounts such that upon admixture of the composition with an aqueous medium in an aqueous medium to composition ratio of about 100:1 by weight, either in vitro or in vivo, a clear aqueous dispersion is formed. Examples of lipid disorders include, without limitation, hypercholesterolemia, hypertriglyceridemia, and mixed dyslipidemia.

The invention additionally provides a method for reducing the dependence of drug absorption on lipolysis for an orally administered therapeutic agent, e.g., a lipid-regulating agent, wherein the method comprises administering the agent in a composition comprising a carrier and a therapeutically effective amount of a therapeutic agent, the carrier including a triglyceride and at least two surfactants, at least one of which is hydrophilic, and, optionally, at least one of which is hydrophobic, wherein the triglyceride and surfactants are selected and present in amounts such that upon admixture of the composition with an aqueous medium in an aqueous medium to composition ratio of about 100:1 by weight, either in vitro or in vivo, a clear aqueous dispersion is formed.

In a related embodiment, the invention provides a method for reducing the dependency of drug absorption on endogenous bile, bile-related patient disease states, or meal fat contents for an orally administered therapeutic agent, e.g., a lipid-regulating agent, wherein the method comprises administering the agent in a composition comprising a carrier and a therapeutically effective amount of a therapeutic agent, the carrier including a triglyceride and at least two surfactants, at least one of which is hydrophilic, and, optionally, at least one of which is hydrophobic, wherein the triglyceride and surfactants are selected and present in amounts such that upon admixture of the composition with an aqueous medium in an aqueous medium to composition ratio of about 100:1 by weight, either in vitro or in vivo, a clear aqueous dispersion is formed.

These and other objects and features of the present invention will become more fully apparent from the following description and appended claims, or may be learned by the practice of the invention as set forth hereinafter.

### DETAILED DESCRIPTION OF THE INVENTION

I. Definitions, Nomenclature and Overview:

In describing and claiming the present invention, the following terminology will be used in accordance with the definitions set out below.

As used in this specification and the appended claims, the singular forms “a,” “an” and “the” include plural referents unless the context clearly dictates otherwise. Thus, for example, reference to “an active agent” includes a single active agent as well a two or more different active agents in combination, reference to “an excipient” includes mixtures of two or more excipients as well as a single excipient, and the like.

The terms “active agent,” “therapeutic agent,” and “drug” are used interchangeably herein to refer to any compound that has a prophylactic or therapeutic effect in the management of a particular disorder. The terms also encompass pharmaceutically acceptable, pharmacologically active derivatives of those active agents specifically mentioned herein, including, but not limited to, salts, esters, amides, prodrugs, active metabolites, isomers, fragments, analogs, complexes, and the like. When the terms “active agent,” “therapeutic agent,” and “drug” are used, then, or when a particular active agent is specifically identified, it is to be understood that applicants are referring not only to the active agent per se but also to pharmaceutically acceptable, pharmacologically active salts, esters, amides, prodrugs, active metabolites, isomers, fragments, analogs, complexes, etc.

The term “dosage form” denotes any form of an orally administrable pharmaceutical composition that contains an amount of active agent sufficient to achieve a therapeutic effect with a single administration. When the dosage form is a tablet or capsule, the dosage is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, “treating” a patient with a compound of the invention includes prevention of a particular lipid disorder in a susceptible individual as well as treatment of a clinically symptomatic individual.

By the terms “effective amount” and “therapeutically effective amount” of a therapeutic agent is meant a nontoxic but sufficient amount of the agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

By a “pharmaceutically acceptable compound” (e.g., an active agent, pharmaceutical carrier, or excipient) is meant a material that is not biologically or otherwise undesirable in an oral dosage form, i.e., the material may be incorporated into a pharmaceutical formulation that when orally administered to a patient does not cause any undesirable biological effects or interact in a deleterious manner with any of the other components of the formulation in which it is contained. Pharmaceutical acceptability of a compound is evidenced by, for example, (1) the presence of the compound in a prescription product that has been approved by the FDA for oral administration, (2) the fact that a particular compound (e.g., a pharmaceutical carrier or excipient) has met the standards of toxicological and manufacturing testing established by the U.S. Food and Drug Administration (FDA) for oral pharmaceutical formulations, or (3) the inclusion of a particular compound on the FDA's Inactive Ingredient Guide for incorporation into an oral pharmaceutical formulation.

By a “pharmaceutically acceptable amount” is meant an amount of a compound (e.g., an active agent, pharmaceutical carrier, or excipient) that is not biologically or otherwise undesirable in an oral dosage form, i.e., the amount of the compound in an orally administered composition or dosage form does not cause any undesirable biological effects. As described above with respect to compounds per se, the pharmaceutical acceptability of an amount of a particular compound may be evidenced by (1) the presence of the amount of the compound in a prescription product that has been approved by the FDA for oral administration, (2) the fact that the amount of the compound in an oral dosage form has met the standards of toxicological and manufacturing testing established by the FDA for oral pharmaceutical formulations, or (3) the presence of the amount of the compound in prescription products that have been approved by the FDA for oral administration.

“Pharmacologically active” (or simply “active”) as in a “pharmacologically active” derivative or analog, refers to a derivative or analog having the same type of pharmacological activity as the parent compound and approximately equivalent in degree.

The present invention overcomes the problems described above characteristic of conventional triglyceride-containing pharmaceutical formulations by providing a unique pharmaceutical composition containing a carrier including triglycerides and a combination of surfactants that can solubilize therapeutically effective amounts of therapeutic agents, including lipid-regulating agents. When the compositions are mixed with an aqueous medium, they are surprisingly able to form homogeneous, single-phase aqueous dispersions that are thermodynamically stable and optically clear. The optical clarity is indicative of a high degree of drug solubilization, which, in turn provides for a substantial increase in the rate and/or extent of absorption, with absorption far less dependent on lipolysis, endogenous bile, bile related patient disease states, or meal fat contents, relative to a corresponding composition that does not give rise to a clear aqueous dispersion upon dilution. Advantageously, the compositions of the present invention are able to increase solubilize greater amounts of triglycerides than conventional compositions, even when the total surfactant concentration is the same as in a conventional composition. The compositions of the present invention are also able to increase the solubilization power of surfactants. These compositions therefore provide for an enhanced extent, rate and/or consistency of absorption of the therapeutic agent.

II. Pharmaceutical Compositions:

A. The Carrier

In one embodiment, the present invention provides an orally administrable pharmaceutical composition including a carrier that comprises a triglyceride and at least two surfactants, at least one of which is a hydrophilic surfactant. The triglyceride and surfactants are present in amounts such that upon dilution with an aqueous medium, either in vitro or in vivo, the composition forms a clear aqueous dispersion. It is a particular and surprising feature of the present invention that the dispersion formed is homogeneous and optically clear, despite the presence of substantial amounts of triglycerides, thereby providing unexpected and significant advantages relative to conventional triglyceride-containing compositions.

Examples of triglycerides suitable for use in the present invention are shown in Table 1. In general, these triglycerides are readily available from commercial sources. For several triglycerides, representative commercial products and/or commercial suppliers are listed.

TABLE 1
TRIGLYCERIDES

| Triglyceride | Commercial Source |
|---|---|
| Aceituno oil | |
| Almond oil | Super Refined Almond Oil |
| | (Croda) |
| Arachis oil | |
| Babassu oil | |
| Blackcurrant seed oil | |
| Borage oil | |
| Buffalo ground oil | |
| Candlenut oil | |
| Canola oil | Lipex 108 (Abitec) |
| Castor oil | |
| Chinese vegetable tallow oil | |
| Cocoa butter | |
| Coconut oil | Pureco 76 (Abitec) |
| Coffee seed oil | |
| Corn oil | Super Refined Corn Oil |
| | (Croda) |
| Cottonseed oil | Super Refined Cottonseed Oil |
| | (Croda) |
| Crambe oil | |
| Cuphea species oil | |
| Evening primrose oil | |
| Grapeseed oil | |
| Groundnut oil | |
| Hemp seed oil | |
| Illipe butter | |
| Kapok seed oil | |
| Linseed oil | |
| Menhaden oil | Super Refined Menhaden Oil |
| | (Croda) |
| Mowrah butter | |
| Mustard seed oil | |
| Oiticica oil | |
| Olive oil | Super Refined Olive Oil |
| | (Croda) |
| Palm oil | |
| Palm kernel oil | |
| Peanut oil | Super Refined Peanut Oil |
| | (Croda) |
| Poppy seed oil | |
| Rapeseed oil | |
| Rice bran oil | |
| Safflower oil | Super Refined Safflower Oil |
| | (Croda) |
| Sal fat | |
| Sesame oil | Super Refined Sesame Oil |
| | (Croda) |
| Shark liver oil | Super Refined Shark Liver Oil |
| | (Croda) |
| Shea nut oil | |
| Soybean oil | Super Refined Soybean Oil |
| | (Croda) |
| Stillingia oil | |
| Sunflower oil | |
| Tall oil | |
| Tea seed oil | |
| Tobacco seed oil | |
| Tung oil (China wood oil) | |
| Uchuba | |
| Vernonia oil | |
| Wheat germ oil | Super Refined Wheat Germ |
| | Oil (Croda) |
| Hydrogenated castor oil | Castorwax |
| Hydrogenated coconut oil | Pureco 100 (Abitec) |
| Hydrogenated cottonseed oil | Dritex C (Abitec) |
| Hydrogenated palm oil | Dritex PST (Abitec); Softisan |
| | 154 (Hüls) |
| Hydrogenated soybean oil | Sterotex HM NF (Abitec); |
| | Dritex S (Abitec) |
| Hydrogenated vegetable oil | Sterotex NF (Abitec); |
| | Hydrokote M (Abitec) |
| Hydrogenated cottonseed and castor oil | Sterotex K (Abitec) |
| Partially hydrogenated soybean oil | Hydrokote APS (Abitec) |
| Partially soy and cottonseed oil | Apex B (Abitec) |
| Glyceryl tributyrate | (Sigma) |
| Glyceryl tricaproate | (Sigma) |
| Glyceryl tricaprylate | (Sigma) |
| Glyceryl tricaprate | Captex 1000 (Abitec) |
| Glyceryl triundecanoate | Captex 8227 (Abitec) |
| Glyceryl trilaurate | (Sigma) |
| Glyceryl trimyristate | Dynasan 114 (Hüls) |
| Glyceryl tripalmitate | Dynasan 116 (Hüls) |
| Glyceryl tristearate | Dynasan 118 (Hüls) |
| Glyceryl triarachidate | (Sigma) |
| Glyceryl trimyristoleate | (Sigma) |
| Glyceryl tripalmitoleate | (Sigma) |
| Glyceryl trioleate | (Sigma) |
| Glyceryl trilinoleate | (Sigma) |
| Glyceryl trilinolenate | (Sigma) |
| Glyceryl tricaprylate/caprate | Captex 300 (Abitec); Captex |
| | 355 (Abitec); Miglyol 810 |
| | (Hüls); Miglyol 812 (Hüls) |
| Glyceryl tricaprylate/caprate/laurate | Captex 350 (Abitec) |
| Glyceryl tricaprylate/caprate/linoleate | Captex 810 (Abitec); Miglyol |
| | 818 (Hüls) |
| Glyceryl tricaprylate/caprate/stearate | Softisan 378 (Hüls); |
| | (Larodan) |
| Glyceryl tricaprylate/laurate/stearate | (Larodan) |
| Glyceryl 1,2-caprylate-3-linoleate | (Larodan) |
| Glyceryl 1,2-caprate-3-stearate | (Larodan) |
| Glyceryl 1,2-laurate-3-myristate | (Larodan) |
| Glyceryl 1,2-myristate-3-laurate | (Larodan) |
| Glyceryl 1,3-palmitate-2-butyrate | (Larodan) |
| Glyceryl 1,3-stearate-2-caprate | (Larodan) |
| Glyceryl 1,2-linoleate-3-caprylate | (Larodan) |

Fractionated triglycerides, modified triglycerides, synthetic triglycerides, and mixtures of triglycerides are also within the scope of the invention.

Preferred triglycerides include vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants,” may be suitable to provide all or part of the triglyceride component for the compositions of the present invention. Examples of commercial surfactant compositions containing triglycerides include some members of the surfactant families Gelucires (Gattefosse), Maisines (Gattefosse), and Imwitors (Hüls). Specific examples of these compositions are:

Gelucire 44/14 (saturated polyglycolized glycerides);

Gelucire 50/13 (saturated polyglycolized glycerides);

Gelucire 53/10 (saturated polyglycolized glycerides);

Gelucire 33/01 (semi-synthetic triglycerides of C₈-C₁₈ saturated fatty acids);

Gelucire 39/01 (semi-synthetic glycerides);

other Gelucires, such as 37/06, 43/01, 35/10, 37/02, 46/07, 48/09, 50/02, 62/05, etc.;

Maisine 35-I (linoleic glycerides); and

Imwitor 742 (caprylic/capric glycerides);

Still other commercial surfactant compositions having significant triglyceride content are known to those skilled in the art. It should be appreciated that such compositions, which contain triglycerides as well as surfactants, may be suitable to provide all or part of the triglyceride component of the compositions of the present invention, as well as all or part of the surfactant component, as described below. Of course, none of the commonly known triglyceride-containing commercial surfactants alone provides the unique pharmaceutical compositions and characteristics as recited in the appended claims.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate. Other preferred triglycerides are saturated polyglycolized glycerides (Gelucire 44/14, Gelucire 50/13 and Gelucire 53/10), linoleic glycerides (Maisine 35-I), and caprylic/capric glycerides (Imwitor 742).

Among the preferred triglycerides, particularly preferred triglycerides include: coconut oil; corn oil; olive oil; palm oil; peanut oil; safflower oil; sesame oil; soybean oil; hydrogenated castor oil; hydrogenated coconut oil; partially hydrogenated soybean oil; glyceryl tricaprate; glyceryl trilaurate; glyceryl trioleate; glyceryl trilinoleate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides (Gelucire 44/14, Gelucire 50/13 and Gelucire 53/10); linoleic glycerides (Maisine 35-I); and caprylic/capric glycerides (Imwitor 742).

Medium chain triglycerides (MCTs) such as glyceryl tricaprylate/caprate are generally most preferred. MCTs, as is understood in the art, are triglyceride compositions that are predominantly composed of C₆-C₁₂ fatty acids. That is, greater than 50% of the total fatty acids in the triglyceride composition have a chain length in the range of six to twelve carbon atoms.

The carrier also includes a combination of surfactants, at least one of which is a hydrophilic surfactant, with the remaining surfactant or surfactants being hydrophilic or hydrophobic. As is well known in the art, the terms “hydrophilic” and “hydrophobic” are relative terms. To function as a surfactant, a compound must necessarily include polar or charged hydrophilic moieties as well as non-polar hydrophobic (lipophilic) moieties; i.e., a surfactant compound must be amphiphilic. An empirical parameter commonly used to characterize the relative hydrophilicity and hydrophobicity of non-ionic amphiphilic compounds is the hydrophilic-lipophilic balance (the “HLB” value). Surfactants with lower HLB values are more hydrophobic, and have greater solubility in oils, whereas surfactants with higher HLB values are more hydrophilic, and have greater solubility in aqueous mediums.

Using HLB values as a rough guide, hydrophilic surfactants are generally considered to be those compounds having an HLB value greater than about 10, as well as anionic, cationic, or zwitterionic compounds for which the HLB scale is not generally applicable. Similarly, hydrophobic surfactants are compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or hydrophobicity for use in the present invention, as described herein.

The carrier of the present invention includes at least one hydrophilic surfactant. The hydrophilic surfactant can be any surfactant suitable for use in pharmaceutical compositions. Suitable hydrophilic surfactants can be anionic, cationic, zwitterionic or non-ionic, although non-ionic hydrophilic surfactants are preferred. In a particularly preferred embodiment, the carrier includes a mixture of two or more hydrophilic surfactants, more preferably two or more non-ionic hydrophilic surfactants. Also preferred are mixtures of at least one hydrophilic surfactant, preferably non-ionic, and at least one hydrophobic surfactant.

The choice of specific surfactants should be made keeping in mind the particular triglycerides and active agent(s) to be used in the composition, and the range of polarity appropriate for the chosen therapeutic agent. With these general principles in mind, a very broad range of surfactants is suitable for use in the present invention. Such surfactants can be grouped into the following general chemical classes detailed in the Tables herein. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the present inventors, is more reliable.

It should be emphasized that the invention is not limited to the surfactants in the Tables, which show representative, but not exclusive, lists of available surfactants.

Polyethoxylated Fatty Acids: Although polyethylene glycol (PEG) itself does not function as a surfactant, a variety of PEG-fatty acid esters have useful surfactant properties. Among the PEG-fatty acid monoesters, esters of lauric acid, oleic acid, and stearic acid are especially useful. Among the surfactants of Table 2, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate, and PEG-20 oleate. Examples of polyethoxylated fatty acid monoester surfactants commercially available are shown in Table 2.

TABLE 2
PEG-FATTY ACID MONOESTER SURFACTANTS

| COMPOUND | COMMERCIAL PRODUCT (SUPPLIER) | HLB |
|---|---|---|
| PEG 4-100 | Crodet L series (Croda) | >9 |
| monolaurate | | |
| PEG 4-100 | Crodet O series (Croda) | >8 |
| monooleate | | |
| PEG 4-100 | Crodet S series (Croda), Myrj Series | >6 |
| monostearate | (Atlas/ICI) | |
| PEG 400 distearate | Cithrol 4DS series (Croda) | >10 |
| PEG 100, 200, 300 | Cithrol ML series (Croda) | >10 |
| monolaurate | | |
| PEG 100, 200, 300 | Cithrol MO series (Croda) | >10 |
| monooleate | | |
| PEG 400 dioleate | Cithrol 4DO series (Croda) | >10 |
| PEG 400-1000 | Cithrol MS series (Croda) | >10 |
| monostearate | | |
| PEG-1 stearate | Nikkol MYS-1EX (Nikko), Coster K1 | 2 |
| | (Condea) | |
| PEG-2 stearate | Nikkol MYS-2 (Nikko) | 4 |
| PEG-2 oleate | Nikkol MYO-2 (Nikko) | 4.5 |
| PEG-4 laurate | Mapeg ® 200 ML (PPG), Kessco ® PEG | 9.3 |
| | 200 ML (Stepan), LIPOPEG 2L | |
| | (LIPO Chem.) | |
| PEG-4 oleate | Mapeg ® 200 MO (PPG), Kessco ® | 8.3 |
| | PEG200 MO (Stepan), | |
| PEG-4 stearate | Kessco ® PEG 200 MS (Stepan), Hodag 20 | 6.5 |
| | S (Calgene), Nikkol MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), Kessco ® | 8.5 |
| | PEG 300 MO (Stepan), Nikkol MYO-6 | |
| | (Nikko), Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), LIPOPEG 4DL | 13 |
| | (Lipo Chem.) | |
| PEG-8 oleate | Mapeg ® 400 MO (PPG), Emulgante A8 | 12 |
| | (Condea); Kessco PEG 400 MO (Stepan) | |
| PEG-8 stearate | Mapeg ® 400 MS (PPG), Myrj 45 | 12 |
| PEG-9 oleate | Emulgante A9 (Condea) | >10 |
| PEG-9 stearate | Cremophor S9 (BASF) | >10 |
| PEG-10 laurate | Nikkol MYL-10 (Nikko), Lauridac 10 | 13 |
| | (Croda) | |
| PEG-10 oleate | Nikkol MYO-10 (Nikko) | 11 |
| PEG-10 stearate | Nikkol MYS-10 (Nikko), Coster K100 | 11 |
| | (Condea) | |
| PEG-12 laurate | Kessco ® PEG 600 ML (Stepan) | 15 |
| PEG-12 oleate | Kessco ® PEG 600 MO (Stepan) | 14 |
| PEG-12 ricinoleate | (CAS #9004-97-1) | >10 |
| PEG-12 stearate | Mapeg ® 600 MS (PPG), Kessco ® | 14 |
| | PEG 600 MS (Stepan) | |
| PEG-15 stearate | Nikkol TMGS-15 (Nikko), Koster K15 | 14 |
| | (Condea) | |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), Kessco ® PEG | 16 |
| | 1000 MS (Stepan), Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet O-100 (Croda) | 18.8 |
| PEG-100 stearate | Myrj 59, Arlacel 165 (ICI) | 19 |
| PEG-200 oleate | Albunol 200 MO (Taiwan Surf.) | >10 |
| PEG-400 oleate | LACTOMUL (Henkel), Albunol 400 MO | >10 |
| | (Taiwan Surf.) | |
| PEG-600 oleate | Albunol 600 MO (Taiwan Surf.) | >10 |

PEG-Fatty Acid Diesters: Polyethylene glycol (PEG) fatty acid diesters are also suitable for use as surfactants in the compositions of the present invention. Among the surfactants in Table 3, preferred hydrophilic surfactants include PEG-20 dilaurate, PEG-20 dioleate, PEG-20 distearate, PEG-32 dilaurate and PEG-32 dioleate. Representative PEG-fatty acid diesters are shown in Table 3.

TABLE 3
PEG-FATTY ACID DIESTER SURFACTANTS

| COMPOUND | COMMERCIAL PRODUCT (SUPPLIER) | HLB |
|---|---|---|
| PEG-4 dilaurate | Mapeg ® 200 DL (PPG), Kessco ® PEG | 7 |
| | 200 DL (Stepan), LIPOPEG 2-DL | |
| | (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), Kessco ® PEG | 11 |
| | 400 DL (Stepan), LIPOPEG 4 DL | |
| | (Lipo Chem.) | |
| PEG-8 dioleate | Mapeg ® 400 DO (PPG), Kessco ® PEG | 8.8 |
| | 400 DO (Stepan), LIPOPEG 4 DO | |
| | (Lipo Chem.) | |
| PEG-8 distearate | Mapeg ® 400 DS (PPG), CDS 400 (Nikkol) | 11 |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), Kessco ® 600 | 10 |
| | DO (Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | >10 |

PEG-Fatty Acid Mono- and Di-ester Mixtures: In general, mixtures of surfactants are also useful in the present invention, including mixtures of two or more commercial surfactant products. Several PEG-fatty acid esters are marketed commercially as mixtures or mono- and diesters. Representative surfactant mixtures are shown in Table 4.

TABLE 4
PEG-FATTY ACID MONO- AND DIESTER MIXTURES

| COMPOUND | COMMERCIAL PRODUCT (SUPPLIER) |
|---|---|
| PEG 4-150 mono, | Kessco ® PEG 200-6000 mono, dilaurate (Stepan) |
| dilaurate | |
| PEG 4-150 mono, | Kessco ® PEG 200-6000 mono, dioleate (Stepan) |
| dioleate | |
| PEG 4-150 mono, | Kessco ® 200-6000 mono, distearate (Stepan) |
| distearate | |

Polyethylene Glycol Glycerol Fatty Acid Esters: Suitable PEG glycerol fatty acid esters are shown in Table 5. Among the surfactants in the Table, preferred hydrophilic surfactants are PEG-glyceryl laurate, PEG-30 glyceryl laurate, PEG-40 glyceryl laurate, PEG-20 glyceryl oleate, and PEG-30 glyceryl oleate.

TABLE 5
PEG GLYCEROL FATTY ACID ESTERS

| COMPOUND | COMMERCIAL PRODUCT (SUPPLIER) | HLB |
|---|---|---|
| PEG-20 glyceryl | Tagat ® L (Goldschmidt) | 16 |
| laurate | | |
| PEG-30 glyceryl | Tagat ® L2 (Goldschmidt) | 16 |
| laurate | | |
| PEG-15 glyceryl | Glycerox L series (Croda) | 15 |
| laurate | | |
| PEG-40 glyceryl | Glycerox L series (Croda) | 15 |
| laurate | | |
| PEG-20 glyceryl | Capmul ® EMG (ABITEC), Aldo ® MS-20 | 13 |
| stearate | KFG (Lonza) | |
| PEG-20 glyceryl | Tagat ® O (Goldschmidt) | >10 |
| oleate | | |
| PEG-30 glyceryl | Tagat ® O2 (Goldschmidt) | >10 |
| oleate | | |

Alcohol-Oil Transesterification Products: A large number of surfactants of different degrees of hydrophobicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol), and PEG-6 caprylic/capric glycerides (Softigen 767). Preferred hydrophobic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40). The latter two surfactants are reported to have HLB values of 10, which is generally considered to be the approximate borderline between hydrophilic and hydrophobic surfactants. For purposes of the present invention, these two surfactants are considered to be hydrophobic. Representative surfactants of this class suitable for use in the present invention are shown in Table 6.

TABLE 6
TRANSESTERIFICATION PRODUCTS OF OILS AND ALCOHOLS

| COMPOUND | COMMERCIAL PRODUCT (SUPPLIER) | HLB |
|---|---|---|
| PEG-3 castor oil | Nikkol CO-3 (Nikko) | 3 |
| PEG-5, 9, and 16 | ACCONON CA series (ABITEC) | 6-7 |
| castor oil | | |
| PEG-20 castor oil | Emalex C-20 (Nihon Emulsion), Nikkol | 11 |
| | CO-20 TX (Nikko) | |
| PEG-23 castor oil | Emulgante EL23 | >10 |
| PEG-30 castor oil | Emalex C-30 (Nihon Emulsion), | 11 |
| | Alkamuls ® EL 620 (Rhone-Poulenc), | |
| | Incrocas 30 (Croda) | |
| PEG-35 castor oil | Cremophor EL and EL-P (BASF), | |
| | Emulphor EL, Incrocas-35 (Croda), | |
| | Emulgin RO 35 (Henkel) | |
| PEG-38 castor oil | Emulgante EL 65 (Condea) | |
| PEG-40 castor oil | Emalex C-40 (Nihon Emulsion), | 13 |
| | Alkamuls ® EL 719 (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated | Nikkol HCO-5 (Nikko) | 6 |
| castor oil | | |
| PEG-7 hydrogenated | Simusol ® 989 (Seppic), Cremophor | 6 |
| castor oil | WO7 (BASF) | |
| PEG-10 hydrogenated | Nikkol HCO-10 (Nikko) | 6.5 |
| castor oil | | |
| PEG-20 hydrogenated | Nikkol HCO-20 (Nikko) | 11 |
| castor oil | | |
| PEG-25 hydrogenated | Simulsol ® 1292 (Seppic), Cerex ELS | 11 |
| castor oil | 250 (Auschem SpA) | |
| PEG-30 hydrogenated | Nikkol HCO-30 (Nikko) | 11 |
| castor oil | | |
| PEG-40 hydrogenated | Cremophor RH 40 (BASF), Croduret | 13 |
| castor oil | (Croda), Emulgin HRE 40 (Henkel) | |
| PEG-45 hydrogenated | Cerex ELS 450 (Auschem Spa) | 14 |
| castor oil | | |
| PEG-50 hydrogenated | Emalex HC-50 (Nihon Emulsion) | 14 |
| castor oil | | |
| PEG-60 hydrogenated | Nikkol HCO-60 (Nikko); Cremophor RH | 15 |
| castor oil | 60 (BASF) | |
| PEG-80 hydrogenated | Nikkol HCO-80 (Nikko) | 15 |
| castor oil | | |
| PEG-100 hydro- | Nikkol HCO-100 (Nikko) | 17 |
| genated castor oil | | |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel | Labrafil ® M 1944 CS (Gattefosse) | 4 |
| oil | | |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated | Labrafil ® M 2130 BS (Gattefosse) | 4 |
| palm kernel oil | | |
| PEG-6 palm kernel | Labrafil ® M 2130 CS (Gattefosse) | 4 |
| oil | | |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn | Crovol M40 (Croda) | 10 |
| glycerides | | |
| PEG-20 almond | Crovol A40 (Croda) | 10 |
| glycerides | | |
| PEG-25 trioleate | TAGAT ® TO (Goldschmidt) | 11 |
| PEG-40 palm kernel | Crovol PK-70 | >10 |
| oil | | |
| PEG-60 corn | Crovol M70 (Croda) | 15 |
| glycerides | | |
| PEG-60 almond | Crovol A70 (Croda) | 15 |
| glycerides | | |
| PEG-4 caprylic/capric | Labrafac ® Hydro (Gattefosse), | 4-5 |
| triglyceride | | |
| PEG-8 caprylic/capric | Labrasol (Gattefosse), Labrafac CM 10 | >10 |
| glycerides | (Gattefosse) | |
| PEG-6 caprylic/capric | SOFTIGEN ® 767 (Hüls), Glycerox 767 | 19 |
| glycerides | (Croda) | |
| Lauroyl macrogol-32 | GELUCIRE 44/14 (Gattefosse) | 14 |
| glyceride | | |
| Stearoyl macrogol | GELUCIRE 50/13 (Gattefosse) | 13 |
| glyceride | | |
| Mono, di, tri, tetra | SorbitoGlyceride (Gattefosse) | <10 |
| esters of vegetable | | |
| oils and sorbitol | | |
| Pentaerythrityl | Crodamol PTIS (Croda) | <10 |
| tetraisostearate | | |
| Pentaerythrityl | Albunol DS (Taiwan Surf.) | <10 |
| distearate | | |
| Pentaerythrityl | Liponate PO-4 (Lipo Chem.) | <10 |
| tetraoleate | | |
| Pentaerythrityl | Liponate PS-4 (Lipo Chem.) | <10 |
| tetrastearate | | |
| Pentaerythrityl | Liponate PE-810 (Lipo Chem.), Crodamol | <10 |
| tetracaprylate/ | PTC (Croda) | |
| tetracaprate | | |
| Pentaerythrityl | Nikkol Pentarate 408 (Nikko) | |
| tetraoctanoate | | |

Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants.

Polyglycerized Fatty Acids: Polyglycerol esters of fatty acids are also suitable surfactants for the present invention. Among the polyglyceryl fatty acid esters, preferred hydrophobic surfactants include polyglyceryl oleate (Plurol Oleique), polyglyceryl-2 dioleate (Nikkol DGDO), and polyglyceryl-10 trioleate. Preferred hydrophilic surfactants include polyglyceryl-10 laurate (Nikkol Decaglyn 1-L), polyglyceryl-10 oleate (Nikkol Decaglyn 1-O), and polyglyceryl-10 mono, dioleate (Caprol® PEG 860). Polyglyceryl polyricinoleates (Polymuls) are also preferred hydrophilic and hydrophobic surfactants. Examples of suitable polyglyceryl esters are shown in Table 7.

TABLE 7
POLYGLYCERIZED FATTY ACIDS

| COMPOUND | COMMERCIAL PRODUCT (SUPPLIER) | HLB |
|---|---|---|
| Polyglyceryl-2 | Nikkol DGMS (Nikko) | 5-7 |
| stearate | | |
| Polyglyceryl-2 | Nikkol DGMO (Nikko) | 5-7 |
| oleate | | |
| Polyglyceryl-2 | Nikkol DGMIS (Nikko) | 5-7 |
| isostearate | | |
| Polyglyceryl-3 | Caprol ® 3GO (ABITEC), Drewpol 3-1-O | 6.5 |
| oleate | (Stepan) | |
| Polyglyceryl-4 | Nikkol Tetraglyn 1-O (Nikko) | 5-7 |
| oleate | | |
| Polyglyceryl-4 | Nikkol Tetraglyn 1-S (Nikko) | 5-6 |
| stearate | | |
| Polyglyceryl-6 | Drewpol 6-1-O (Stepan), Nikkol Hexaglyn | 9 |
| oleate | 1-O (Nikko) | |
| Polyglyceryl-10 | Nikkol Decaglyn 1-L (Nikko) | 15 |
| laurate | | |
| Polyglyceryl-10 | Nikkol Decaglyn 1-O (Nikko) | 14 |
| oleate | | |
| Polyglyceryl-10 | Nikkol Decaglyn 1-S (Nikko) | 12 |
| stearate | | |
| Polyglyceryl-6 | Nikkol Hexaglyn PR-15 (Nikko) | >8 |
| ricinoleate | | |
| Polyglyceryl-10 | Nikkol Decaglyn 1-LN (Nikko) | 12 |
| linoleate | | |
| Polyglyceryl-6 | Nikkol Hexaglyn 5-O (Nikko) | <10 |
| pentaoleate | | |
| Polyglyceryl-3 | Cremophor GO32 (BASF) | <10 |
| dioleate | | |
| Polyglyceryl-3 | Cremophor GS32 (BASF) | <10 |
| distearate | | |
| Polyglyceryl-4 | Nikkol Tetraglyn 5-O (Nikko) | <10 |
| pentaoleate | | |
| Polyglyceryl-6 | Caprol ® 6G20 (ABITEC); Hodag PGO-62 | 8.5 |
| dioleate | (Calgene), PLUROL OLEIQUE CC 497 | |
| | (Gattefosse) | |
| Polyglyceryl-2 | Nikkol DGDO (Nikko) | 7 |
| dioleate | | |
| Polyglyceryl-10 | Nikkol Decaglyn 3-O (Nikko) | 7 |
| trioleate | | |
| Polyglyceryl-10 | Nikkol Decaglyn 5-O (Nikko) | 3.5 |
| pentaoleate | | |
| Polyglyceryl-10 | Nikkol Decaglyn 7-O (Nikko) | 3 |
| septaoleate | | |
| Polyglyceryl-10 | Caprol ® 10G4O (ABITEC); Hodag | 6.2 |
| tetraoleate | PGO-62 (CALGENE), Drewpol 10-4-O | |
| | (Stepan) | |
| Polyglyceryl-10 | Nikkol Decaglyn 10-IS (Nikko) | <10 |
| decaisostearate | | |
| Polyglyceryl-101 | Drewpol 10-10-O (Stepan), Caprol 10G10O | 3.5 |
| decaoleate | (ABITEC), Nikkol Decaglyn 10-O | |
| Polyglyceryl-10 | Caprol ® PGE 860 (ABITEC) | 11 |
| mono, dioleate | | |
| Polyglyceryl | Polymuls (Henkel) | 3-20 |
| polyricinoleate | | |

Propylene Glycol Fatty Acid Esters: Esters of propylene glycol and fatty acids are suitable surfactants for use in the present invention. In this surfactant class, preferred hydrophobic surfactants include propylene glycol monolaurate (Lauroglycol FCC), propylene glycol ricinoleate (Propymuls), propylene glycol monooleate (Myverol P-O6), and propylene glycol dioctanoate (Captex® 800). Examples of surfactants of this class are given in Table 8.

TABLE 8
PROPYLENE GLYCOL FATTY ACID ESTERS

| COMPOUND | COMMERCIAL PRODUCT (SUPPLIER) | HLB |
|---|---|---|
| Propylene glycol | Capryol 90 (Gattefosse), Nikkol Sefsol 218 | <10 |
| monocaprylate | (Nikko) | |
| Propylene glycol | Lauroglycol 90 (Gattefosse), Lauroglycol FCC | <10 |
| monolaurate | (Gattefosse) | |
| Propylene glycol | Lutrol OP2000 (BASF) | <10 |
| oleate | | |
| Propylene glycol | Mirpyl | <10 |
| myristate | | |
| Propylene glycol | ADM PGME-03 (ADM), LIPO PGMS (Lipo | 3-4 |
| monostearate | Chem.), Aldo ® PGHMS (Lonza) | |
| Propylene glycol | | <10 |
| hydroxy stearate | | |
| Propylene glycol | PROPYMULS (Henkel) | <10 |
| ricinoleate | | |
| Propylene glycol | | <10 |
| isostearate | | |
| Propylene glycol | Myverol P-O6 (Eastman) | <10 |
| monooleate | | |
| Propylene glycol | Captex ® 800 (ABITEC) | >6 |
| dioctanoate | | |
| Propylene glycol | LABRAFAC PG (Gattefosse) | >6 |
| caprylate/caprate | | |
| Propylene glycol | | >6 |
| dilaurate | | |
| Propylene glycol | Kessco ® PGDS (Stepan) | >6 |
| distearate | | |
| Propylene glycol | Nikkol Sefsol 228 (Nikko) | >6 |
| dicaprylate | | |
| Propylene glycol | Nikkol PDD (Nikko) | >6 |
| dicaprate | | |

Mixtures of Propylene Glycol Esters—Glycerol Esters: In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel 186). Examples of these surfactants are shown in Table 9.

TABLE 9
GLYCEROL/PROPYLENE GLYCOL FATTY ACID ESTERS

| COMPOUND | COMMERCIAL PRODUCT (SUPPLIER) | HLB |
|---|---|---|
| Oleic | ATMOS 300, ARLACEL 186 (ICI) | 3-4 |
| Stearic | ATMOS 150 | 3-4 |

Mono- and Diglycerides: A particularly important class of surfactants is the class of mono- and diglycerides. These surfactants are generally hydrophobic. Preferred hydrophobic surfactants in this class of compounds include glyceryl monooleate (Peceol), glyceryl ricinoleate, glyceryl laurate, glyceryl dilaurate (Capmul® GDL), glyceryl dioleate (Capmul® GDO), glyceryl mono/dioleate (Capmul® GMO-K), glyceryl caprylate/caprate (Capmul® MCM), caprylic acid mono/diglycerides (Imwitor® 988), and mono- and diacetylated monoglycerides (Myvacet® 9-45). Examples of these surfactants are given in Table 10.

TABLE 10
MONO- AND DIGLYCERIDE SURFACTANTS

| COMPOUND | COMMERCIAL PRODUCT (SUPPLIER) | HLB |
|---|---|---|
| Monopalmitolein | (Larodan) | <10 |
| (C16:1) | | |
| Monoelaidin | (Larodan) | <10 |
| (C18:1) | | |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl | Nikkol MGM (Nikko) | 3-4 |
| monomyristate | | |
| (C14) | | |
| Glyceryl | PECEOL (Gattefosse), Hodag GMO-D, | 3-4 |
| monooleate (C18:1) | Nikkol MGO (Nikko) | |
| Glyceryl | RYLO series (Danisco), DIMODAN | 3-4 |
| monooleate | series (Danisco), EMULDAN (Danisco), | |
| | ALDO ® MO FG (Lonza), Kessco GMO | |
| | (Stepan), MONOMULS ® series | |
| | (Henkel), TEGIN O, DREWMULSE | |
| | GMO (Stepan), Atlas G-695 (ICI), | |
| | GMOrphic 80 (Eastman), ADM DMG-40, | |
| | 70, and 100 (ADM), Myverol (Eastman) | |
| Glycerol | OLICINE (Gattefosse) | 3-4 |
| monooleate/ | | |
| linoleate | | |
| Glycerol | Maisine (Gattefosse), MYVEROL 18-92, | 3-4 |
| monolinoleate | Myverol 18-06 (Eastman) | |
| Glyceryl ricinoleate | Softigen ® 701 (Hüls), HODAG GMR-D | 6 |
| | (Calgene), ALDO ® MR (Lonza) | |
| Glyceryl | ALDO ® MLD (Lonza), Hodag GML | 6.8 |
| monolaurate | (Calgene) | |
| Glycerol | Emalex GMS-P (Nihon) | 4 |
| monopalmitate | | |
| Glycerol | Capmul ® GMS (ABITEC), Myvaplex | 5-9 |
| monostearate | (Eastman), IMWITOR ® 191 (Hüls), | |
| | CUTINA GMS, Aldo ® MS (Lonza), | |
| | Nikkol MGS series (Nikko) | |
| Glyceryl mono-, | Capmul ® GMO-K (ABITEC) | <10 |
| dioleate | | |
| Glyceryl palmitic/ | CUTIINA MD-A, ESTAGEL-G18 | <10 |
| stearic | | |
| Glyceryl acetate | Lamegin ® EE (Grünau GmbH) | <10 |
| Glyceryl laurate | Imwitor ® 312 (Hüls), Monomuls ® | 4 |
| | 90-45 (Grünau GmbH), Aldo ® MLD | |
| | (Lonza) | |
| Glyceryl citrate/ | Imwitor ® 375 (Hüls) | <10 |
| lactate/oleate/ | | |
| linoleate | | |
| Glyceryl caprylate | Imwitor ® 308 (Hüls), Capmul ® | 5-6 |
| | MCMC8 (ABITEC) | |
| Glyceryl caprylate/ | Capmul ® MCM (ABITEC) | 5-6 |
| caprate | | |
| Caprylic acid | Imwitor ® 988 (Hüls) | 5-6 |
| mono, diglycerides | | |
| Caprylic/capric | Imwitor ® 742 (Hüls) | <10 |
| glycerides | | |
| Mono- and | Myvacet ® 9-45, Myvacet ® | 3.8-4 |
| diacetylated | 9-40, Myvacet ® 9-08 (Eastman), | |
| monoglycerides | Lamegin ® (Grünau) | |
| Glyceryl | Aldo ® MS, Arlacel 129 (ICI), LIPO | 4.4 |
| monostearate | GMS (Lipo Chem.), Imwitor ® 191 | |
| | (Hüls), Myvaplex (Eastman) | |
| Lactic acid esters of | LAMEGIN GLP (Henkel) | <10 |
| mono, diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | <10 |
| Distearin | (Larodan) | <10 |
| Glyceryl | Capmul ® GDL (ABITEC) | 3-4 |
| dilaurate (C12) | | |
| Glyceryl dioleate | Capmul ® GDO (ABITEC) | 3-4 |
| Glycerol esters of | GELUCIRE 39/01 (Gattefosse), | 1 |
| fatty acids | GELUCIRE 43/01 (Gattefosse) | |
| | GELUCIRE 37/06 (Gattefosse) | 6 |
| Dipalmitolein | (Larodan) | <10 |
| (C16:1) | | |
| 1,2 and 1,3-diolein | (Larodan) | <10 |
| (C18:1) | | |
| Dielaidin (C18:1) | (Larodan) | <10 |
| Dilinolein (C18:2) | (Larodan) | <10 |

Sterol and Sterol Derivatives: Sterols and derivatives of sterols are suitable surfactants for use in the present invention. These surfactants can be hydrophilic or hydrophobic. Preferred derivatives include the polyethylene glycol derivatives. A preferred hydrophobic surfactant in this class is cholesterol. A preferred hydrophilic surfactant in this class is PEG-24 cholesterol ether (Solulan C-24). Examples of surfactants of this class are shown in Table 11.

TABLE 11
STEROL AND STEROL DERIVATIVE SURFACTANTS

| | COMMERCIAL | |
|---|---|---|
| COMPOUND | PRODUCT (SUPPLIER) | HLB |
| Cholesterol, sitosterol, lanosterol | | <10 |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-5 (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

Polyethylene Glycol Sorbitan Fatty Acid Esters: A variety of PEG-sorbitan fatty acid esters are available and are suitable for use as surfactants in the present invention. In general, these surfactants are hydrophilic, although several hydrophobic surfactants of this class can be used. Among the PEG-sorbitan fatty acid esters, preferred hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80). Examples of these surfactants are shown in Table 12.

TABLE 12
PEG-SORBITAN FATTY ACID ESTERS

| COMPOUND | COMMERCIAL PRODUCT (SUPPLIER) | HLB |
|---|---|---|
| PEG-10 sorbitan | Liposorb L-10 (Lipo Chem.) | >10 |
| laurate | | |
| PEG-20 sorbitan | Tween-20 (Atlas/ICI), Crillet 1 (Croda), | 17 |
| monolaurate | DACOL MLS 20 (Condea) | |
| PEG-4 sorbitan | Tween-21 (Atlas/ICI), Crillet 11 (Croda) | 13 |
| monolaurate | | |
| PEG-80 sorbitan | Hodag PSML-80 (Calgene); T-Maz 28 | >10 |
| monolaurate | | |
| PEG-6 sorbitan | Nikkol GL-1 (Nikko) | 16 |
| monolaurate | | |
| PEG-20 sorbitan | Tween-40 (Atlas/ICI), Crillet 2 (Croda) | 16 |
| monopalmitate | | |
| PEG-20 sorbitan | Tween-60 (Atlas/ICI), Crillet 3 (Croda) | 15 |
| monostearate | | |
| PEG-4 sorbitan | Tween-61 (Atlas/ICI), Crillet 31 (Croda) | 9.6 |
| monostearate | | |
| PEG-8 sorbitan | DACOL MSS (Condea) | >10 |
| monostearate | | |
| PEG-6 sorbitan | Nikkol TS106 (Nikko) | 11 |
| monostearate | | |
| PEG-20 sorbitan | Tween-65 (Atlas/ICI), Crillet 35 (Croda) | 11 |
| tristearate | | |
| PEG-6 sorbitan | Nikkol GS-6 (Nikko) | 3 |
| tetrastearate | | |
| PEG-60 sorbitan | Nikkol GS-460 (Nikko) | 13 |
| tetrastearate | | |
| PEG-5 sorbitan | Tween-81 (Atlas/ICI), Crillet 41 (Croda) | 10 |
| monooleate | | |
| PEG-6 sorbitan | Nikkol TO-106 (Nikko) | 10 |
| monooleate | | |
| PEG-20 sorbitan | Tween-80 (Atlas/ICI), Crillet 4 (Croda) | 15 |
| monooleate | | |
| PEG-40 sorbitan | Emalex ET 8040 (Nihon Emulsion) | 18 |
| oleate | | |
| PEG-20 sorbitan | Tween-85 (Atlas/ICI), Crillet 45 (Croda) | 11 |
| trioleate | | |
| PEG-6 sorbitan | Nikkol GO-4 (Nikko) | 8.5 |
| tetraoleate | | |
| PEG-30 sorbitan | Nikkol GO-430 (Nikko) | 12 |
| tetraoleate | | |
| PEG-40 sorbitan | Nikkol GO-440 (Nikko) | 13 |
| tetraoleate | | |
| PEG-20 sorbitan | Tween-120 (Atlas/ICI), Crillet 6 (Croda) | >10 |
| monoisostearate | | |
| PEG sorbitol | Atlas G-1086 (ICI) | 10 |
| hexaoleate | | |
| PEG-6 sorbitol | Nikkol GS-6 (Nikko) | 3 |
| hexastearate | | |

Polyethylene glycol alkyl ethers: Ethers of polyethylene glycol and alkyl alcohols are suitable surfactants for use in the present invention. Preferred hydrophobic ethers include PEG-3 oleyl ether (Volpo 3) and PEG-4 lauryl ether (Brij 30). Examples of these surfactants are shown in Table 13.

TABLE 13
POLYETHYLENE GLYCOL ALKYL ETHERS

| | COMMERCIAL | |
|---|---|---|
| COMPOUND | PRODUCT (SUPPLIER) | HLB |
| PEG-2 oleyl ether, oleth-2 | Brij 92/93 (Atlas/ICI) | 4.9 |
| PEG-3 oleyl ether, oleth-3 | Volpo 3 (Croda) | <10 |
| PEG-5 oleyl ether, oleth-5 | Volpo 5 (Croda) | <10 |
| PEG-10 oleyl ether, oleth-10 | Volpo 10 (Croda), Brij 96/97 | 12 |
| | (Atlas/ICI) | |
| PEG-20 oleyl ether, oleth-20 | Volpo 20 (Croda), Brij 98/99 | 15 |
| | (Atlas/ICI) | |
| PEG-4 lauryl ether, laureth-4 | Brij 30 (Atlas/ICI) | 9.7 |
| PEG-9 lauryl ether | | >10 |
| PEG-23 lauryl ether, laureth-23 | Brij 35 (Atlas/ICI) | 17 |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

Sugar Esters: Esters of sugars are suitable surfactants for use in the present invention. Preferred hydrophilic surfactants in this class include sucrose monopalmitate and sucrose monolaurate. Examples of such surfactants are shown in Table 14.

TABLE 14
SUGAR ESTER SURFACTANTS

| | COMMERCIAL | |
|---|---|---|
| COMPOUND | PRODUCT (SUPPLIER) | HLB |
| Sucrose distearate | SUCRO ESTER 7 (Gattefosse), | 3 |
| | Crodesta F-10 (Croda) | |
| Sucrose distearate/ | SUCRO ESTER 11 (Gattefosse), | 12 |
| monostearate | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate 1695 | 15 |
| | (Mitsubishi-Kasei) | |

Polyethylene Glycol Alkyl Phenols: Several hydrophilic PEG-alkyl phenol surfactants are available, and are suitable for use in the present invention. Examples of these surfactants are shown in Table 15.

TABLE 15
Polyethylene Glycol Alkyl Phenol Surfactants

| COMPOUND | COMMERCIAL PRODUCT (SUPPLIER) | HLB |
|---|---|---|
| PEG-10-100 nonyl | Triton X series (Rohm & Haas), Igepal CA | >10 |
| phenol | series (GAF, USA), Antarox CA series | |
| | (GAF, UK) | |
| PEG-15-100 octyl | Triton N-series (Rohm & Haas), Igepal CO | >10 |
| phenol ether | series (GAF, USA), Antarox CO series | |
| | (GAF, UK) | |

Polyoxyethylene-Polyoxypropylene Block Copolymers: The POE-POP block copolymers are a unique class of polymeric surfactants. The unique structure of the surfactants, with hydrophilic POE and hydrophobic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. The generic term for these polymers is “poloxamer” (CAS 9003-11-6). These polymers have the formula HO(C₂H₄O)ₐ(C₃H₆O)_{b}(C₂H₄O)ₐH where “a” and “b” denote the number of polyoxyethylene and polyoxypropylene units, respectively.

Preferred hydrophilic surfactants of this class include Poloxamers 108, 188, 217, 238, 288, 338, and 407. Preferred hydrophobic surfactants in this class include Poloxamers 124, 182, 183, 212, 331, and 335.

Examples of suitable surfactants of this class are shown in Table 16. Since the compounds are widely available, commercial sources are not listed in the Table. The compounds are listed by generic name, with the corresponding “a” and “b” values.

TABLE 16
POE-POP BLOCK COPOLYMERS

| | | |
|---|---|---|
| | A, B VALUES IN | |
| COMPOUND | HO(C₂H₄O)_{A}(C₃H₆O)_{B}(C₂H₄O)_{A}H | HLB |

| | | | |
|---|---|---|---|
| Poloxamer 105 | a = 11 | b = 16 | 8 |
| Poloxamer 108 | a = 46 | b = 16 | >10 |
| Poloxamer 122 | a = 5 | b = 21 | 3 |
| Poloxamer 123 | a = 27 | b = 21 | 7 |
| Poloxamer 124 | a = 11 | b = 21 | >7 |
| Poloxamer 181 | a = 3 | b = 30 | |
| Poloxamer 182 | a = 8 | b = 30 | 2 |
| Poloxamer 183 | a = 10 | b = 30 | |
| Poloxamer 184 | a = 13 | b = 30 | |
| Poloxamer 185 | a = 19 | b = 30 | |
| Poloxamer 188 | a = 75 | b = 30 | 29 |
| Poloxamer 212 | a = 8 | b = 35 | |
| Poloxamer 215 | a = 24 | b = 35 | |
| Poloxamer 217 | a = 52 | b = 35 | |
| Poloxamer 231 | a = 16 | b = 39 | |
| Poloxamer 234 | a = 22 | b = 39 | |
| Poloxamer 235 | a = 27 | b = 39 | |
| Poloxamer 237 | a = 62 | b = 39 | 24 |
| Poloxamer 238 | a = 97 | b = 39 | |
| Poloxamer 282 | a = 10 | b = 47 | |
| Poloxamer 284 | a = 21 | b = 47 | |
| Poloxamer 288 | a = 122 | b = 47 | >10 |
| Poloxamer 331 | a = 7 | b = 54 | 0.5 |
| Poloxamer 333 | a = 20 | b = 54 | |
| Poloxamer 334 | a = 31 | b = 54 | |
| Poloxamer 335 | a = 38 | b = 54 | |
| Poloxamer 338 | a = 128 | b = 54 | |
| Poloxamer 401 | a = 6 | b = 67 | |
| Poloxamer 402 | a = 13 | b = 67 | |
| Poloxamer 403 | a = 21 | b = 67 | |
| Poloxamer 407 | a = 98 | b = 67 | |

Sorbitan Fatty Acid Esters: Sorbitan esters of fatty acids are suitable surfactants for use in the present invention. Among these esters, preferred hydrophobic surfactants include sorbitan monolaurate (Arlacel 20), sorbitan monopalmitate (Span-40), sorbitan monooleate (Span-80), sorbitan monostearate, and sorbitan tristearate. Examples of these surfactants are shown in Table 17.

TABLE 17
SORBITAN FATTY ACID ESTER SURFACTANTS

| COMPOUND | COMMERCIAL PRODUCT (SUPPLIER) | HLB |
|---|---|---|
| Sorbitan | Span-20 (Atlas/ICI), Crill 1 (Croda), Arlacel 20 | 8.6 |
| monolaurate | (ICI) | |
| Sorbitan | Span-40 (Atlas/ICI), Crill 2 (Croda), Nikkol | 6.7 |
| monopalmitate | SP-10 (Nikko) | |
| Sorbitan | Span-80 (Atlas/ICI), Crill 4 (Croda), Crill 50 | 4.3 |
| monooleate | (Croda) | |
| Sorbitan | Span-60 (Atlas/ICI), Crill 3 (Croda), Nikkol | 4.7 |
| monostearate | SS-10 (Nikko) | |
| Sorbitan | Span-85 (Atlas/ICI), Crill 45 (Croda), Nikkol | 4.3 |
| trioleate | SO-30 (Nikko) | |
| Sorbitan | Arlacel-C (ICI), Crill 43 (Croda), Nikkol | 3.7 |
| sesquioleate | SO-15 (Nikko) | |
| Sorbitan | Span-65 (Atlas/ICI) Crill 35 (Croda), Nikkol | 2.1 |
| tristearate | 55-30 (Nikko) | |
| Sorbitan | Crill 6 (Croda), Nikkol SI-10 (Nikko) | 4.7 |
| monoisostearate | | |
| Sorbitan | Nikkol SS-15 (Nikko) | 4.2 |
| sesquistearate | | |

Lower Alcohol Fatty Acid Esters: Esters of lower alcohols (C₂ to C₄) and fatty acids (C₈ to C₁₈) are suitable surfactants for use in the present invention. Among these esters, preferred hydrophobic surfactants include ethyl oleate (Crodamol EO), isopropyl myristate (Crodamol IPM), and isopropyl palmitate (Crodamol IPP). Examples of these surfactants are shown in Table 18.

TABLE 18
LOWER ALCOHOL FATTY ACID ESTER SURFACTANTS

| COMPOUND | COMMERCIAL PRODUCT (SUPPLIER) | HLB |
|---|---|---|
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

Ionic Surfactants: Ionic surfactants, including cationic, anionic and zwitterionic surfactants, are suitable hydrophilic surfactants for use in the present invention. Preferred anionic surfactants include fatty acid salts and bile salts. Preferred cationic surfactants include carnitines. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Examples of such surfactants are shown in Table 19. For simplicity, typical counterions are shown in the entries in the Table. It will be appreciated by one skilled in the art, however, that any bioacceptable counterion may be used. For example, although the fatty acids are shown as sodium salts, other cation counterions can also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in the Table.

TABLE 19
TONIC SURFACTANTS

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glycochenodeoxycholate | |
| Sodium cholylsarcosinate | |
| Sodium N-methyl taurocholate | |
| Sodium lithocholate | |
| PHOSPHOLIPIDS | |
| Egg/Soy lecithin [Epikuron ™ (Lucas Meyer), | |
| Ovothin ™ (Lucas Meyer)] | |
| Lyso egg/soy lecithin | |
| Hydroxylated lecithin | |
| Lysophosphatidyleholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl | |
| ether phosphate | |
| Esterification products of fatty alcohols or fatty | |
| alcohol ethoxylates with phosphoric acid or | |
| anhydride | |
| CARBOXYLATES | |
| Ether carboxylates (by oxidation of terminal OH | |
| group of fatty alcohol ethoxylates) | |
| Succinylated monoglycerides [LAMEGIN ZE | |
| (Henkel)] | |
| Sodium stearyl fumarate | |
| Stearoyl propylene glycol hydrogen succinate | |
| Mono/diacetylated tartaric acid esters of mono- | |
| and diglycerides | |
| Citric acid esters of mono-, diglycerides | |
| Glyceryl-lacto esters of fatty acids | |
| (CFR ref. 172.852) | |
| Acyl lactylates: | |
| lactylic esters of fatty acids | |
| calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamido-MEA-sulfosuccinate | |
| CATIONIC Surfactants | >10 |
| Lauroyl carnitine | |
| Palmitoyl carnitine | |
| Myristoyl carnitine | |
| Hexadecyl triammonium bromide | |
| Dodecyl ammonium chloride | |
| Alkyl benzyldimethylammonium salts | |
| Diisobutyl phenoxyethoxydimethyl | |
| benzylammonium salts | |
| Alkylpyridinium salts | |
| Betaines (trialkylglycine): | |
| Lauryl betaine (N-lauryl,N,N-dimethylglycine) | |
| Ethoxylated amines: | |
| Polyoxyethylene-15 coconut amine | |

Unionized Ionizable Surfactants: Ionizable surfactants, when present in their unionized (neutral, non-salt) form, are hydrophobic surfactants suitable for use in the compositions and methods of the present invention. Particular examples of such surfactants include free fatty acids, particularly C₆-C₂₂ fatty acids, and bile acids. More specifically, suitable unionized ionizable surfactants include the free fatty acid and bile acid forms of any of the fatty acid salts and bile salts shown in Table 19.

Preferred Surfactants and Surfactant Combinations: Among the above-listed surfactants, several combinations are preferred. In all of the preferred combinations, the carrier includes at least one hydrophilic surfactant. Preferred non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; and mixtures thereof.

More preferably, the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. The glyceride can be a monoglyceride, diglyceride, triglyceride, or a mixture.

Also preferred are non-ionic hydrophilic surfactants that are reaction mixtures of polyols and fatty acids, glycerides, vegetable oils, hydrogenated vegetable oils or sterols. These reaction mixtures are largely composed of the transesterification products of the reaction, along with often complex mixtures of other reaction products. The polyol is preferably glycerol, ethylene glycol, polyethylene glycol, sorbitol, propylene glycol, pentaerythritol, or a saccharide.

Several particularly preferred carrier compositions are those which include as a non-ionic hydrophilic surfactant PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, or a poloxamer.

Among these preferred surfactants, more preferred are PEG-20 laurate, PEG-20 oleate, PEG-35 castor oil, PEG-40 palm kernel oil, PEG-40 hydrogenated castor oil, PEG-60 corn oil, PEG-25 glyceryl trioleate, polyglyceryl-10 laurate, PEG-6 caprate/caprylate glycerides, PEG-8 caprate/caprylate glycerides, PEG-30 cholesterol, polysorbate 20, polysorbate 80, POE-9 lauryl ether, POE-23 lauryl ether, POE-10 oleyl ether, PEG-24 cholesterol, sucrose monostearate, sucrose monolaurate and poloxamers. Most preferred are PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, and hydrophilic poloxamers.

The hydrophilic surfactant can also be, or include as a component, an ionic surfactant. Preferred ionic surfactants include alkyl ammonium salts; bile acids and salts, analogues, and derivatives thereof; fusidic acid and derivatives thereof, fatty acid conjugates of amino acids, oligopeptides, and polypeptides; glyceride esters of amino acids, oligopeptides, and polypeptides; acyl lactylates; mono- and diacetylated tartaric acid esters of mono- and diglycerides; succinylated monoglycerides; citric acid esters of mono- and diglycerides; alginate salts; propylene glycol alginate; lecithins and hydrogenated lecithins; lysolecithin and hydrogenated lysolecithins; lysophospholipids and derivatives thereof, phospholipids and derivatives thereof, salts of alkylsulfates; salts of fatty acids; sodium docusate; carnitines; and mixtures thereof.

More preferable ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono- and diacetylated tartaric acid esters of mono- and diglycerides; succinylated monoglycerides; citric acid esters of mono- and diglycerides; carnitines; and mixtures thereof.

More specifically, preferred ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Particularly preferred ionic surfactants are lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

The carrier of the present compositions includes at least two surfactants, at least one of which is hydrophilic. In one embodiment, the present invention includes at two surfactants that are hydrophilic, and preferred hydrophilic surfactants are listed above. In another embodiment, the carrier includes at least one hydrophilic surfactant and at least one hydrophobic surfactant. In general, the particularly preferred hydrophilic surfactants are selected from polyoxyethylene sorbitan fatty acids esters, polyoxyethylene vegetable oils, polyoxyethylene hydrogenated vegetable oils, and mixtures thereof, and hydrophilic transesterification products of oils (including oil-soluble vitamins), with the most preferred hydrophilic surfactants being polysorbate 80, PEG-35 castor oil, PEG-40 castor oil, and tocopheryl PEG-1000 succinate.

In those compositions that contain a hydrophobic surfactant, exemplary hydrophobic surfactants include, without limitation: alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils.

As with the hydrophilic surfactants, hydrophobic surfactants can be hydrophobic transesterification products of polyols and fatty acids, glycerides, vegetable oils, hydrogenated vegetable oils, and sterols.

Preferably, the hydrophobic surfactant is selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid esters of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols.

More preferred are lower alcohol fatty acids esters; polypropylene glycol fatty acid esters; propylene glycol fatty acid esters; glycerol fatty acid esters; acetylated glycerol fatty acid esters; lactic acid esters of mono/diglycerides; sorbitan fatty acid esters; polyoxyethylene vegetable oils; and mixtures thereof, with glycerol fatty acid esters and acetylated glycerol fatty acid esters being most preferred. Among the glycerol fatty acid esters, the esters are preferably mono- or diglycerides, or mixtures of mono- and diglycerides, where the fatty acid moiety is a C₆ to C₂₂ fatty acid.

Also preferred are hydrophobic surfactants that are the reaction mixture of polyols and fatty acids, glycerides, vegetable oils, hydrogenated vegetable oils, and sterols. Preferred polyols are polyethylene glycol, sorbitol, propylene glycol, and pentaerythritol.

Specifically preferred hydrophobic surfactants include myristic acid; oleic acid; lauric acid; stearic acid; palmitic acid; PEG 1-4 stearate; PEG 2-4 oleate; PEG-4 dilaurate; PEG-4 dioleate; PEG-4 distearate; PEG-6 dioleate; PEG-6 distearate; PEG-8 dioleate; PEG 3-16 castor oil; PEG 5-10 hydrogenated castor oil; PEG 6-20 corn oil; PEG 6-20 almond oil; PEG-6 olive oil; PEG-6 peanut oil; PEG-6 palm kernel oil; PEG-6 hydrogenated palm kernel oil; PEG-4 capric/caprylic triglyceride, mono, di, tri, tetra esters of vegetable oil and sorbitol; pentaerythrityl di, tetra stearate, isostearate, oleate, caprylate, or caprate; polyglyceryl 2-4 oleate, stearate, or isostearate; polyglyceryl 4-10 pentaoleate; polyglyceryl-3 dioleate; polyglyceryl-6 dioleate; polyglyceryl-10 trioleate; polyglyceryl-3 distearate; propylene glycol mono- or diesters of a C₆ to C₂₀ fatty acid; monoglycerides of C₆ to C₂₀ fatty acids; acetylated monoglycerides of C₆ to C₂₀ fatty acids; diglycerides of C₆ to C₂₀ fatty acids; lactic acid derivatives of monoglycerides; lactic acid derivatives of diglycerides; cholesterol; phytosterol; PEG 5-20 soya sterol; PEG-6 sorbitan tetra, hexastearate; PEG-6 sorbitan tetraoleate; sorbitan monolaurate; sorbitan monopalmitate; sorbitan mono, trioleate; sorbitan mono, tristearate; sorbitan monoisostearate; sorbitan sesquioleate; sorbitan sesquistearate; PEG 2-5 oleyl ether; POE 2-4 lauryl ether; PEG-2 cetyl ether; PEG-2 stearyl ether; sucrose distearate; sucrose dipalmitate; ethyl oleate; isopropyl myristate; isopropyl palmitate; ethyl linoleate; isopropyl linoleate; and poloxamers.

The more preferred hydrophobic surfactants are as follows: oleic acid; lauric acid; glycerol linoleate, glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; and poloxamers. Of these, the most preferred hydrophobic surfactants are PEG-6 corn oil, PEG-6 apricot kernel oil, and mixtures thereof.

B. Therapeutic Agents

Therapeutic agents that may be administered using the compositions and methods of the invention include both hydrophobic agents and hydrophilic agents, although the former are generally preferred.

The preferred class of drugs to be administered using the compositions and methods of the invention are lipid-regulating agents, i.e., any agents that have a prophylactic or therapeutic effect when administered to a patient susceptible to or suffering from a lipid disorder. Lipid disorders, include, by way of example, hypercholesterolemia, hypertriglyceridemia, and mixed dyslipidemia. In a preferred embodiment, the lipid-regulating agent is one that is capable of being solubilized in at least one of the components of the composition, i.e., in the triglyceride, the surfactants, or both the triglyceride and the surfactants.

One type of lipid-regulating agent is a cholesterol-lowering agent, i.e., an active agent that when administered to a human subject who has or is predisposed to hypercholesterolemia, has the effect of beneficially modifying serum cholesterol levels. More particularly, cholesterol-lowering agents lower serum low density lipoprotein (LDL) cholesterol levels or inhibit oxidation of LDL cholesterol, whereas high density lipoprotein (HDL) serum cholesterol levels may be lowered, remain the same, or be increased. Preferred cholesterol-lowering agents are fibric acid derivatives, HMG CoA reductase inhibitors, bile acid sequestrants, and probucol. Cholesterol-lowering agents are well known in the art and are discussed and reviewed in numerous publications; a useful review is presented by Witztum, J. L., “Drugs used in the treatment of hyperlipidemia”, in Hardman, J. G., Gilman, A. G., and Limbird, L. E., editors, Goodman and Gilman's The Pharmacological Basis of Therapeutics, 9^{th} Edition, pp. 875-897 (New York: McGraw-Hill, 1996). Brief descriptions of some of the classes of cholesterol-lowering agents that may be used in this invention follow.

Fibric acid derivatives: These compounds, also known as “fibrates,” lower triglyceride levels, raise high density lipoprotein (HDL) levels, and have variable effects on LDL cholesterol levels in the blood. These compounds act by inhibiting the synthesis and secretion of triglycerides in the liver and activating a lipoprotein lipase. Examples of fibric acid derivatives that may be used in this invention include, without limitation, bezafibrate, beclobrate, binifibrate, ciprofibrate, clinofibrate, clofibrate, etofibrate, fenofibrate, gemfibrozil, nicofibrate, pirifibrate, ronifibrate, simfibrate, and theofibrate, and the corresponding acids (e.g., clofibric acid, fenofibric acid, etc.).

HMG CoA reductase inhibitors: The members of this class of compounds inhibit 3-hydroxy-3-methylglutaryl coenzyme A (HMG CoA) reductase. This enzyme catalyzes the conversion of HMG CoA to mevalonate, which is an early and rate-limiting step in the biosynthesis of cholesterol. Examples of HMG CoA reductase inhibitors that may be used include but are not limited to lovastatin (MEVACOR™; see U.S. Pat. No. 4,231,938), simvastatin (ZOCOR™; see U.S. Pat. No. 4,444,784), pravastatin (PRAVACHOL™; see U.S. Pat. No. 4,346,227), fluvastatin (LESCOL™; see U.S. Pat. No. 5,354,772), atorvastatin (LIPITOR™; see U.S. Pat. No. 5,273,995), cerivastatin (also called rivastatin; see U.S. Pat. No. 5,177,080), mevastatin (see U.S. Pat. No. 3,883140), fluindostatin (Sandoz XU-62-320), nystatin, pitivastatin, rosuvastatin, urinastatin, velostatin (also called synvinolin; see U.S. Pat. Nos. 4,448,784 and 4,450,171), and compounds related to these as described in the cited references. Some other examples of HMG CoA reductase inhibitors that may be used are, without limitation, those presented in U.S. Pat. No. 6,264,938 at Table 1 and U.S. Pat. No. 5,622,985, columns 3 through 6. Other compounds that inhibit the activity of HMG CoA reductase can be readily identified by using assays well known in the art; see, as examples, the assays described or cited in U.S. Pat. No. 4,231,938 at column 6, and in International Patent Publication WO 84/02131 at pp. 30-33. The term “HMG CoA reductase inhibitor” is intended to include all pharmaceutically acceptable salt, ester, lactone forms, and other derivatives of compounds that have HMG CoA reductase inhibitory activity, and therefore the use of such salt, ester, lactone forms, and other derivatives is included within the scope of this invention.

Bile acid sequestrants: Bile acids, which are secreted into the intestine to aid in the digestion and absorption of lipids, are synthesized in the liver from cholesterol. Normally, approximately 97% of bile acids are reabsorbed and reused. If large amounts of bile acids are excreted, then the liver must convert more cholesterol to bile acids, lowering serum cholesterol levels, particularly LDL cholesterol levels. Although biosynthesis of cholesterol is up-regulated in this case, the net effect of increased bile acid synthesis in most individuals is to lower cholesterol, particularly LDL cholesterol, levels in the serum. Generally, bile acid sequestrants are poorly absorbed resins or other substances that bind to and sequester bile acids in the intestine. The sequestered bile acids are subsequently excreted in the feces. Any bile acid sequestrant may be used in the practice of this invention. Examples of suitable bile acid sequestrants that may be used in this invention include, without limitation, cholestyramine, colesevelam, colestipol, poly[methyl-(3-trimethylaminopropyl)imino-trimethylene dihalide], and those agents disclosed in U.S. Pat. No. 6,271,264, International Patent Publication WO 95/34585, and European Patent Publication No. EP 0 622,078.

Probucol: This compound is a potent lipophilic antioxidant that inhibits the oxidation of LDL cholesterol. As the oxidation of LDL cholesterol may be an important, and perhaps essential, factor in the development of atherosclerotic lesions, probucol may be useful in preventing or treating atherosclerosis. Although probucol is known to lower serum cholesterol levels, the mechanism of action is not well understood. Probucol is often useful in treating patients who do not respond to other cholesterol-lowering drugs, such as patients with homozygous familial hypercholesterolemia.

Anti-diabetic agents: Anti-diabetic agents that may be advantageously administered using the methods and compositions of the invention include compounds in the following classes: sulfonyl ureas, biguanides, thiazolidinediones, alpha glucosidase inhibitors, and meglitinides. Specific examples of anti-diabetic agents include, without limitation, glyburide, metformin, glipizide, glimepiride, pioglitazone, rosiglitazone, miglitol, acarbose, repaglinide, nateglinide, and mixtures thereof

Anti-hypertensive agents: Representative anti-hypertensive agents include amlodipine, benazepril, benidipine, candesartan, captopril, carvedilol, darodipine, diltiazem, diazoxide, doxazosin, enalapril, epleronone, eposartan, felodipine, fenoldopam, fosinopril, guanabenz, iloprost, imidapril, irbesartan, isradipine, lercardinipine, lisinopril, losartan, mibefradil, minoxidil, nebivolol, nicardipine, nifedipine, nimodipine, nisoldipine, olmesartan, omapatrilat, phenoxybenzamine, pindolol, prazosin, quinapril, reserpine, semotiadil, sitaxsentan, terazosin, telmisartan, trandolapril, and valsartan.

Still other lipid-regulating agents include, without limitation: squalene synthesis inhibitors such as (S)-alpha-bis(2,2-dimethyl-1-oxopropoxy)methoxy-phosphinyl-3-phenoxybenzenebutanesulfonic acid mono potassium salt (BMS-188494); LDL catabolism enhancers such as flufenamic acid and indomethacin; steroid (e.g., cholesterol) absorption inhibitors, such as ezemitibe; nicotinic acid and derivatives such as nicomol and niceritrol.

In a preferred embodiment, the lipid-regulating agent is a fibric acid derivative, with fenofibrate particularly preferred. In this embodiment, it must be noted that the absorption of fenofibrate following administration of a composition of the invention is not dependent on the dissolution of fenofibrate in the patient's gastrointestinal tract since a substantial fraction of the fenofibrate in the composition itself is already solubilized. Thus, it is not necessary to micronize the drug prior to incorporation into the present compositions. To the extent that fenofibrate is micronized in the present compositions, e.g., to further enhance solubility, it is preferred that the fenofibrate is micronized in the absence of any other components, particularly solid surfactants.

Any of the aforementioned lipid-regulating agents may be administered in combination. Furthermore, any of the aforementioned lipid-regulating agents may be co-administered with an additional active agent, which may or may not be contained within the same composition or dosage form. It is particularly preferred that any additional such active agent also serve as a lipid-regulating agent. The weight ratio of the active agents co-administered may be varied and will depend upon the effective dose of each ingredient.

Additional active agents may be solubilized or suspended with or without the presence of an additional solubilizer. For those additional active agents that are ionized or ionizable, the compositions described herein may include a buffer to facilitate or maintain the presence of a preferred ionized form of the additional active agent in the composition.

In the preferred embodiment, wherein the lipid-regulating agent is fenofibrate, the preferred active agents that are co-administered therewith are those that bind cholesterol, e.g., cholestyramine, to synergistically treat certain lipid disorders. Other preferred additional active agents for co-administration with fenofibrate include acipimox, acifran, p-aminosalicylic acid, aspirin, colestipol, fluindostatin, fluvastatin, gemfibrozil, imanixil, istigmastanyl phosphorylcholine, lipostabil, lovastatin, melinamide, mevastatin, neomycin, nicotinic acid, probucol, tetrahydrolipostatin, rapamycin, progesterone, estradiol, captopril, pivopril, enalopril, fosinopril, ramipril, cetapril, cilazapril, delapril, indolapril, spirapril, quinapril and mixtures thereof.

Still other examples of additional active agents include: insulin sensitivity enhancers, insulin secretion enhancers and/or an insulin preparation; α-glucosidase inhibitors; aldose reductase inhibitors; biguanides; and angiotensin converting enzyme (ACE) inhibitors.

Insulin sensitivity enhancers are agents that substantially increase insulin sensitivity in muscle, liver and adipose tissue resulting in the correction of elevated plasma levels of glucose, triglycerides and nonesterified fatty acids without the occurrence of hypoglycemia. Examples of insulin sensitivity enhancers include, but are not limited to, the glitazones (thiazolidinediones such as pioglitazone, troglitazone, rosiglitazone, MCC-555, and BRL49653).

Insulin secretion enhancers are drugs that promote the secretion of insulin from pancreatic beta-cells. The group of drugs known as sulfonylureas represents a preferred class of insulin secretion enhancers. The sulfonylureas are drugs that promote the secretion of insulin from pancreatic beta-cells by transmitting signals of insulin secretion via sulfonylurea receptors in cell membranes. Examples of the sulfonylureas include, but are not limited to: tolbutamide; chlorpropamide; tolazamide; acetohexamide; 4-chloro-N→(1-pyrolidinylamino)carbonyl-benzenesulfonamide (generic name: glycopyramide) or its ammonium salt; glibenclamide (glyburide); gliclazide; 1-butyl-3-metanilylurea; carbutamide; glibonuride; glipizide; gliquidone; glisoxepid; glybuthiazole; glibuzole; glyhexarmide; glymidine; glypinamide; phenbutamide; tolcyclanide and combinations thereof. Other insulin secretion enhancers include N-(4-(1-methylethyl)cyclohexyl)carbonyl-D-phenylalanine (AY-4166), calcium(2S)-2-benzyl-3-(cis-hexahydro-2-isoindolinylcarbonyl)propionate dihydrate (KAD-1229), and glimepiride (Hoe 490).

Examples of insulin preparations include animal insulin preparations typically extracted from bovine or porcine pancreas and human insulin preparations synthesized by genetic engineering techniques typically using Escherichia coli or yeasts. Each of these types of insulin is readily available commercially from, for example, Eli Lilly and Co., Indianapolis, Ind. While insulin preparations are available in a variety of types, e.g. immediate-acting, bimodal-acting, intermediate-acting, and long-acting, these types of preparations can be selectively administered according to the patient's condition.

α-Glucosidase inhibitors are drugs that inhibit digestive enzymes such as amylase, maltase, α-dextrinase, sucrase, etc. to retard digestion of starch and sugars. Examples preferred α-glucosidase inhibitors include acarbose, N-(1,3-dihydroxy-2-propyl)valiolamine (generic name; voglibose), miglitol and combinations thereof. Voglibose is a particularly preferred α-glucosidase inhibitor.

Aldose reductase inhibitors are drugs that inhibit the first-stage rate-limiting enzyme in the polyol pathway to prevent or arrest diabetic complications. In the hyperglycemic state of diabetes, the utilization of glucose in the polyol pathway is increased and the excess sorbitol accumulated intracellularly acts as a tissue toxin. The toxicity triggers the onset of complications such as diabetic neuropathy, retinopathy and nephropathy. Examples of aldose reductase inhibitors include, but are not limited to, tolurestat; epalrestat, imirestat, zenarestat, zopolrestat, sorbinil; 1-(3-bromo-2-benzofuranyl)sulfonyl-2,4-imidazolidinedione (M-16209) and combinations thereof

Biguanides are drugs that stimulate anaerobic glycolysis, increase the sensitivity to insulin in the peripheral tissues, inhibit glucose absorption from the intestine, suppress hepatic gluconeogenesis, and inhibit fatty acid oxidation. Examples of the biguanides include, but are not limited to phenformin, metformin, buformin and combinations thereof.

ACE inhibitors are drugs that partially lower blood glucose levels in addition to lowering blood pressure by inhibiting angiotensin converting enzymes. Most of these compounds can be classified into three groups based on their chemical structure: (1) sulfhydryl-containing ACE inhibitors, including captopril and agents that are structurally related to captopril, such as fentiapril, pivalopril, zofenopril and alacepril; (2) dicarboxyl-containing ACE inhibitors, including enalapril and agents that are structurally related to enalapril, such as lisinopril, benazepril, quinapril, moxipril, ramipril, spirapril, perindopril, indolapril, pentopril, indalapril and cilazapril; and (3) phosphorus-containing ACE inhibitors, structurally related to fosinopril. Many of the ACE inhibitors are esters developed for high oral bioavailability, but with low potency in themselves; they must be converted to particular metabolites in the body that have potent activity. Some further examples of ACE inhibitors that may be used in the practice of this invention are, without limitation, AB-103, ancovenin, benazeprilat, BRL-36378, BW-A575C, CGS13928C, CL242817, CV-5975, Equaten, EU-4865, EU-4867, EU-5476, foroxymithine, FPL 66564, FR-900456, Hoe-065, 15B2, indolapril, ketomethylureas, KRI-1177, KRI-1230, L681176, libenzapril, MCD, MDL-27088, MDL-27467A, moveltipril, MS-41, nicotianamine, pentopril, phenacetin, pivopril, renatapril, RG-5975, RG-6134, RG-6207, RGH0399, ROO-911, RS-10085-197, RS-2039, RS 5139, RS-86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y23785, Yissum, P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-66564, idrapril, perindoprilat, and Servier S-5590.

More generally, therapeutic agents that may be administered using the methods and compositions of the invention include, without limitation, the following:

ketodesogestrel, 4-dihydrotestosterone, abecarnil, acamprostate, acavir, acebutolol, aceclofenac, acemetacin, acetaminophen, acetaminosalol, acetanilide, acetohexamide, acetophenazine maleate, acetophenazine, acetoxolone, acetoxypregnenolone, acetretin, acrisorcin, acrivastine, acyclovir, adinazolam, adiphenine hydrochloride, adrafinil, adrenolone, agatroban, ahnitrine, akatinol, alatrofloxacin, albendazole, albuterol, aldioxa, alendronate, alfentanil, alibendol, alitretinoin, allopurinol, allylamines, allylestrenol, alminoprofen, almotriptan, alosetron, aloxiprin, alprazolam, alprenolol, amantadine, ambucetamide, amidephrine, amidinomycin, amiloride, aminoarylcarboxylic acid derivatives, arminoglutethimide, aminoglycosides, aminopentamide, aminopromazine, aminorex, amiodarone, amiphenazole, amiprilose, amisulpride, amitriptyline, amlexanox, amiodipine, amodiaquine, amosulalol, amotriphene, amoxapine, amoxicillin, amphecloral, amphetamine, amphomycin, amphotericin, ampicillin, ampiroxicam, amprenavir, amrinone, amsacrine, amyl nitrate, amylobarbitone, anagestone acetate, anastrozole, amdinocillin, androstenediol, androstenediol-17-acetate, androstenediol-17-benzoate, androstenediol-3-acetate, androstenediol-3-acetate-17-benzoate, androstenedione, androsterone acetate, androsterone benzoate, androsterone propionate, androsterone, angiotensin, anidulafungin, aniracetam, apazone, apicycline, apoatropine, apomorphine, apraclonidine, aprepitant, aprotinin, arbaprostil, ardeparin, aripiprazole, amikacin, arotinolol, arsthinol, arylacetic acid derivatives, arylalkylamines, arylbutyric acid derivatives, arylcarboxylic acids, arylpiperazines, arylpropionic acid derivatives, aspirin, astemizole, atenolol, atomoxetine, atorvastatin, atovaquone, atropine, auranofin, azapropazone, azathioprine, azelastine, azetazolamide, azithromycin, baclofen, bambuterol, bamethan, barbitone, bamidipine, basalazide, beclamide, beclobrate, beclomethasone, befimolol, bemegride, benazepril, bencyclane, bendazac, bendazol, bendroflumethiazide, benethamine penicillin, benexate hydrochloride, benfurodil hemisuccinate, benidipine, benorylate, bentazepam, benzhexol, benziodarone, benznidazole, benzoctamine, benzodiazepine derivatives, benzodiazepine, benzonatate, benzphetamine, benzylmorphine, beperiden, bephenium hydroxynaphthoate, bepridil, betahistine, betamethasone, betaxolol, bevantolol, bevonium methyl sulfate, bexarotene, bezafibrate, bialamicol, biapenem, bicalutamide, bietamiverine, bifonazole, binedaline, binifibrate, biricodar, bisacodyl, bisantrene, bisoprolol, bitolterol, bopindolol, boswellic acid, bradykinin, bretylium, bromazepam, bromocriptine, bromperidol, brotizolam, brovincamine, bucloxic acid, bucumolol, budesonide, budralazine, bufeniode, bufetolol, buflomedil, bufuralol, bumetanide, bunitrolol, bupranolol, buprenorphine, buproprion, buspirone, busulfan, butalamine, butaverine, butenafine, butidrine hydrochloride, butobarbitone, butoconazole nitrate, butoconazole, butofilolol, butorphenol, butropium bromide, cabergoline, calcifediol, calcipotriene, calcitriol, caldiribine, cambendazole, camioxirole, camostat, camptothecin, candesartan, candoxatril, capecitabine, capsaicin, captopril, carazolol, carbacephems, carbamates, carbamazepine, carbapenem, carbarsone, carbatrol, carbenoxolone, carbimazole, carbromal, carbuterol, carisoprodol, carotenes, caroverine, carteolol, carvedilol, cefaclor, cefazolin, cefbuperazone, cefepime, cefoselis, ceftibuten, celecoxib, celiprolol, cephaeline, cephalosporin C, cephalosporins, cephamycins, cerivastatin, certoparin, cetamolol, cetiedil, cetirizine, cetraxate, chloracizine, chlorambucil, chlorbetamide, chlordantoin, chlordiazepoxide, chlormadinone acetate, chlormethiazole, chloroquine, chlorothiazide, chlorpheniramine, chlorphenoxamide, chlorphentermine, chlorproguanil, chlorpromazine, chlorpropamide, chlorprothixene, chlortetracycline, chlorthalidone, cholecalciferol, chromonar, ciclesonide, ciclonicate, cidofivir, ciglitazone, cilansetron, cilostazol, cimetidine, cimetropium bromide, cinepazet maleate, cinnamedrine, cinnarizine, cinolazepam, cinoxacin, ciprofibrate, ciprofloxacin, cisapride, cisplatin, citalopram, citicoline, clarithromycin, clebopride, clemastine, clenbuterol, clidanac, clinofibrate, clioquinol, clobazam, clobenfurol, clobenzorex, clofazimine, clofibrate, clofibric acid, cloforex, clomipramine, clonazepam, clonidine, clonitrate, clopidogrel, clopirac indomethacin, cloranolol, cloricromen, clorprenaline, clortermine, clotiazepam, clotrimazole, cloxacillin, clozapine, cinepazide, codeine methyl bromide, codeine phosphate, codeine sulfate, codeine, colloidal bismuth subcitrate, cortisone, cromafiban, cromolyn, cropropamide, crotethamide, curcumin, cyclandelate, cyclarbamate, cyclazocine, cyclexedrine, cyclizine, cyclobenzaprine, cyclodrine, cyclonium iodide, cyclopentamine, cyclosporin, cyproheptadine, cyproterone acetate, cyproterone, cytarabine, dacarbazine, dalfopristine, dantrolene sodium, dapiprazole, darodipine, decitabine, decoquinate, dehydroemetine, dehydroepiandrosterone, delavirdine, delaviridine, demeclocycline, denopamine, deramciclane, descitalopram, desipramine, desloratadine, desogestrel, desomorphine, desoxymethasone, detomidine, dexamethasone, dexamphetamine, dexanabinol, dexchlorpheniramine, dexfenfluramine, dexmethylphenidate, dexrazoxane, dextroamphetamine sulfate, dextroamphetamine, dextropropoxyphene, diamorphine, diazemine, diazepam, diazoxide, dibromopropamidine, dichlorophen, diclofenac, dicumarol, didanosine, dideoxyadenosine, diethylpropion, difemerine, difenamizole, diflunisal, digitoxin, digoxin, dihydroergotamine, dihydrocodeine, dihydrocodeinone enol acetate, dihydroergotamine mesylate, dihydroergotamine, dihydrogesterone, dihydromorphine, dihydropyridine derivatives, dihydrostreptomycin, dihydrotachysterol, dihydroxyaluminum acetylsalicylate, diiodohydroxyquinoline, diisopromine, dilazep, dilevalol, dilitazem, diloxanide furoate, diloxanide, diltiazem, dimefline, dimenhydrinate, dimethisterone, dimetofrine, dimorpholamine, dinitolmide, dioxaphetyl butyrate, dioxethedrine, diphemethoxidine, diphenhydramine, diphenoxylate, diphetarsone, dipivefrin, diponium bromide, dipyridamole, dirithromycin, disopyramide, divalproex sodium, dofetilide, domperidone, donezepil, dopexamine, dopradil, dosmalfate, doxapram, doxazosin, doxefazepam, doxepin, doxycycline, drofenine, dromostanolone propionate, dromostanolone, dronabinol, droperidol, droprenilamine, duloxetine, dutasteride, ebrotidine, eburnamonine, ecabet, ecenofloxacin, econazole nitrate, edavarone, edoxudine, efavirenz, efloxate, eledoisin, eletriptan, elgodipine, ellipticine, emepronium bromide, emetine, enalapril, encainide, enloplatin, enoximone, enprostil, entacapone, epanolol, ephedrine, epinastine, epinephrine, epirubicin, epleronone, eposartan, ergocalciferol, ergoloid mesylates, ergotamine, ertapenum, erythromycin, erythrityl tetranitrate, esaprazole, escitalopram, esmolol, esomeprazole, esonarimod, estazolam, estradiol benzoate, estradiol, estramustine, estriol succinate, estriol, estrone acetate, estrone sulfate, etafedrine, etafenone, ethacrynic acid, ethamivan, ethinamate, ethinylestradiol 3-acetate, ethinylestradiol 3-benzoate, ethinylestradiol, ethionamide, ethisterone (17α-ethinyltestosterone), ethopropazine, ethotoin, ethoxyphenamine, ethylestrenol, ethylmorphine, ethylnorepinephrine, ethynodiol diacetate, etodolac, etofibrate, etoposide, etoricoxib, etretinate, everolimus, exalamide, exemestane, examorelin, ezemitibe, falecalcitriol, famciclovir, famotidine, fantofarone, farapenum, farglitazar, fasudil, felbamate, felodipine, fenalamide, fenbufen, fenbutrazate, fendiline, fenfluramine, fenofibrate, fenofibric acid, fenoldopam, fenoprofen, fenoterol, fenoverine, fenoxazoline, fenoxedil, fenpiprane, fenproporex, fenspiride, fentanyl, fexofenadine, flavoxate, flecainide, flopropione, floredil, floxuridine, fluconazole, flucytosine, fludarabine, fludiazepam, fludrocortisone, flufenamic acid, flunanisone, flunarizine, flunisolide, flunitrazepam, fluocortolone, fluoxetine, flupenthixol decanoate, fluphenazine decanoate, fluphenazine enanthate, fluphenazine, fluproquazone, flurazepam, flurbiprofen, flurogestone acetate, fluticasone propionate, fluvastatin, fluvoxamine, fominoben, formoterol, foscarnet, foscarnet, fosinopril, fosphenytoin, frovatriptan, fudosteine, fumagillin, furazolidone, furfurylmethyl amphetamine, furosemide, gabapentin, gabexate, gaboxadol, galanthamine, gallopamil, gammaparin, ganciclovir, ganglefene, gefarnate, gemcitabine, gemfibrozil, gepirone, gestadene, ghrelin, glatiramer, glaucarubin, glibenclamide, gliclazide, glimepiride, glipizide, gluconic acid, glutamic acid, glyburide, glyceryl trinitrate, granisetron, grepafloxacin, griseofulvin, guaiazulene, guanabenz, guanfacine, halofantrine, haloperidol decanoate, haloperidol, haloxazolam, hepronicate, hexobendine, hexoprenaline, hydramitrazine, hydrazides, hydrochlorothiazide, hydrocodone, hydrocortisone, hydromorphone, hydroxyamphetamine, hydroxymethylprogesterone acetate, hydroxymethylprogesterone, hydroxyprogesterone acetate, hydroxyprogesterone caproate, hydroxyprogesterone, hymecromone, hyoscyamine, ibopamine, ibudilast, ibufenac, ibuprofen, ibutilide, idebenone, idoxuridine, ifenprodil, igmesine, iloprost, imatinib, imidapril, imidazoles, imipenem, imipramine, imolamine, incadronic acid pergolide, indanazoline, indenolol, indinavir, indomethacin, indoramin, inosine pranobex, inositol niacinate, iodoquinol, ipidacrine, iproniazid, irbesartan, irinotecan, irsogladine, isoetharine, isometheptene, isoproterenol, isosorbide dinitrate, isosorbide mononitrate, isoxsuprine, isrodipine, itasetron, itraconazole, itramin tosylate, ivermectin, kallidin, kallikrein, kanamycin, ketamine, ketoconazole, ketoprofen, ketorolac, ketotifen, labetalol, lafutidine, lamifiban, lamivudine, lamotrigine, lanatoside c, lansoprazole, lasofoxifene, leflunomide, leminoprazole, lercanidipine, lesopitron, letrozole, leucovorin, levalbuterol, levallorphan, levetiracetam, levobunolol, levodopa, levofloxacin, levonorgestrel, levophacetoperane, levorphanol, lidocaine, lidoflazine, lifibrol, limaprost, linezolid, lintitript, liranaftate, lisinopril, lisuride, lobeline, lobucavir, lodoxamide, lomefloxacin, lomerizine, lomustine, loperamide, lopinavir, loprazolam, loracarbef, loratadine, lorazepam, lorefloxacin, lormetazepam, losartan, lovasatain, loxapine succinate, loxapine, 1-threo-methylphenidate, lumiracoxib, lynestrenol, lysine acetylsalicylate, lysozyme, lysuride, mabuterol, mafenide, magnesium acetylsalicylate, malgramostin, mannitol hexanitrate, maprotiline, mazindol, mebendazole, meclizine, meclofenamic acid, mecloxamine pentapiperide, medazepam, medibazine, medigoxin, medrogestone, medroxyprogesterone acetate, mefenamic acid, mefenorex, mefloquine, megestrol acetate, megestrol, melengestrol acetate, melphalan, mematine, mepenzolate bromide, meperidine, mephenoxalone, mephentermine, mepindolol, mepixanox, meprobamate, meptazinol, mercaptopurine, merropenum, mesalamine, mesalazine, mesoridazine besylate, mesoridazine, mestranol, metaclazepam, metamfepramone, metampicillin, metaproterenol, metaraminol, methacycline, methadone hydrochloride, methadone, methamphetamine, methaqualone, methoin, methotrexate, methoxamine, methsuximide, methylhexaneamine, methylphenidate, methylphenobarbitone, methylprednisolone, methysergide, metiazinic acid, metizoline, metoclopramide, metolazone, metoprolol, metoxalone, metripranolol, metronidazole, mexiletine, mianserin, mibefradil, miconazole, midazolam, midodrine, miglitol, milnacipran, milrinone, minoxidil, mirtazapine, misoprostol, mitomycin, mitotane, mitoxantrone, mizolastine, modafinil, mofebutazone, mofetil, molindone hydrochloride, molindone, molsidomine, monatepil, montelukast, monteplase, moprolol, moricizine, morphine hydrochloride, morphine sulfate, morphine, morpholine salicylate, mosapramine, moxifloxacin, moxisylyte, moxonidine, mycophenolate, nabumetone, nadolol, nadoxolol, nadroparin, nafamostat, nafronyl, naftopidil, nalbuphine, nalidixic acid, nalmefene, nalorphine, naloxone, naltrexone, nandrolone benzoate, nandrolone cyclohexanecarboxylate, nandrolone cyclohexane-propionate, nandrolone decanoate, nandrolone furylpropionate, nandrolone phenpropionate, naphazoline, naproxen, naratriptan, natamycin, nateglinide, nebivalol, nedocromil, nefazodone, nefopam, nelfinavir, nemonapride, neomycin undecylenate, neomycin, neotrofin, nesiritide, n-ethylamphetamine, nevibulol, nevirapine, nexopamil, nicametate, nicardipine, nicergoline, nicofibrate, nicofuranose, nicomorphine, nicorandil, nicotinyl alcohol, nicoumalone, nifedipine, nifenalol, nikethamide, nilutamide, nilvadipine, nimodipine, nimorazole, nipradilol, nisoldipine, nitisonone, nitrazepam, nitrofurantoin, nitrofurazone, nitroglycerin, nizatidine, norastemizole, norepinephrine, norethindrone acetate, norethindrone, norethisterone acetate, norethisterone, norethynodrel, norfenefrine, norfloxacin, norgestimate, norgestrel, norgestrienone, normethadone, normethisterone, normorphine, norpseudoephedrine, nortriptyline, novantrone, nylidrin, nystatin, octamylamine, octodrine, octopamine, ofloxacin, olanzapine, olapatadine, olmesartan, olopatidine, olsalazine, omapatrilat, omeprazole, ondansetron, opium, oprelvekin, orlistat, ornidazole, ornoprostil, oseltamivir, oxalatoplatin, oxamniquine, oxandrolone, oxantel embonate, oxaprozin, oxatomide pemirolast, oxatomide, oxazepam, oxcarbazepine, oxfendazole, oxiconazole, oxiracetam, oxolinicacid, oxprenolol, oxycodone, oxyfedrine, oxymetazoline, oxymorphone, oxyphenbutazone, oxyphencyclimine, oxyprenolol, ozagrel, paclitaxel, palonosetron, pantoprazole, papaverine, paracalcitol, paramethadione, parecoxib, pariprazole, paromomycin, paroxetine, parsalmide, pazinaclone, pemoline, penbutolol, penciclovir, penicillin G benzathine, penicillin G procaine, penicillin V, penicillins, pentaerythritol tetranitrate, pentapiperide, pentazocine, pentifylline, pentigetide, pentobarbitone, pentorex, pentoxifylline, pentrinitrol, perbuterol, pergolide, perhexiline, perindopril erbumine, perospirone, perphenazine pimozide, perphenazine, phanquinone, phenacemide, phenacetin, phenazopyridine, phencarbamide, phendimetrazine, phenelzine, phenindione, phenmetrazine, phenobarbitone, phenoperidine, phenothiazines, phenoxybenzamine, phensuximide, phentermine, phentolamine, phenyl salicylate, phenylacetate, phenylbutazone, phenylephrine hydrochloride, phenylpropanolamine hydrochloride, phenylpropanolamine hydrochloride, phenylpropylmethylamine, phenytoin, phloroglucinol, pholedrine, physostigmine salicylate, physostigmine, phytonadiol, piapenum, picilorex, piclamilast, picrotoxin, picumast, pifamine, pilsicainide, pimagedine, pimeclone, pimecrolimus, pimefylline, pimozide, pinaverium bromide, pindolol, pioglitazone, piperacillin, piperazine estrone sulfate, piperazine derivatives, piperilate, piracetam, pirbuterol, pirenzepine, piribedil, pirifibrate, piroxicam, pitavastatin, pizotyline, plaunotol, polaprezinc, polybenzarsol, polyestrol phosphate, practolol, pralnacasan, pramipexole, pranlukast, prasterone, pravastatin, prazepam, praziquantel, prazosin, prednisolone, prednisone, pregabalin, prenalterol, prenylamine, pridinol, prifinium bromide, primidone, primipramine, probenecid, probucol, procainamide, procarbazine, procaterol, prochlorperazine, progesterone, proguanil, pronethalol, propafenone, propamidine, propatyl nitrate, propentoffyline, propionate, propiram, propoxyphene, propranolol, propylhexedrine, propylthiouracil, protokylol, protriptyline, proxazole, pseudoephedrine, purines, pyrantel embonate, pyrazoles, pyrazolones, pyridofylline, pyrimethamine, pyrimidines, pyrrolidones, quazepam, quetiapine, quinagolide, quinapril, quinestrol, quinfamide, quinidine, quinine sulfate, quinolones, quinupristin, rabeprazole sodium, rabeprazole, racefimine, ramatroban, ramipril, ranitidine, ranolazine, ransoprazole, rasagiline, rebamipide, refludan, repaglinide, repinotan, repirinast, reproterol, reserpine, retinoids, ribavirin, rifabutine, rifampicin, rifapentine, rilmenidine, riluzole, rimantadine, rimiterol, rioprostil, risperidone, ritanovir, ritapentine, ritipenem, ritodrine, ritonavir, rivastigmine, rizatriptan, robalzotan, rociverine, rofecoxib, rohypnol, rolipram, romoxipride, ronifibrate, ropinirole, ropivacaine, rosaprostol, rosiglitazone, rosuvastatin, rotinolol, rotraxate, roxatidine acetate, roxindole, rubitecan, salacetamide, salicin, salicylamide, salicylic acid derivatives, salmeterol, saquinavir, saquinavir, scopolamine, secnidazole, selegiline, semotiadil, seratrodast, sertindole, sertraline, sibutramine, sildenafil, simfibrate, simvastatin, siramesine, sirolimus, sitaxsentan, sofalcone, somotiadil, sorivudine, sotalol, soterenol, sparfloxacin, spasmolytol, spectinomycin, spiramycin, spironolactone, spizofurone, stanozolol, stavudine, streptomycin, succinylsulfathiazole, sucralfate, sufentanil, sulconazole nitrate, sulfacetamide, sulfadiazine, sulfaloxicacid, sulfarside, sulfinalol, sulindac, suloctidil, sulphabenzamide, sulphacetamide, sulphadiazine, sulphadoxine, sulphafurazole, sulphamerazine, sulphamethoxazole, sulphapyridine, sulphasalazine, sulphinpyrazone, sulpiride, sulthiame, sultopride, sultroponium, sumanirole, sumatriptan, sunepitron, superoxide dismutase, suplatast, suramin sodium, synephrine, tacrine, tacrolimus, tadalafil, talinolol, talipexole, tamoxifen, tamsulosin, targretin, tazanolast, tazarotene, tazobactum, tecastimezole, teclozan, tedisamil, tegaserod, telenzepine, telmisartan, temazepam, teniposide, teprenone, terazosin, terbinafine, terbutaline sulfate, terbutaline, terconazole, terfenadine, terodiline, terofenamate, tertatolol, testolactone, testosterone, tetracyclics, tetracycline, tetrahydrocannabinol, tetrahydrozoline, thalidomide, theofibrate, thiabendazole, thiazinecarboxamides, thiocarbamates, thiocarbamizine, thiocarbarsone, thioridazine, thiothixene, tiagabine, tiamenidine, tianeptine, tiaprofenic acid, tiaramide, ticlopidine, tigloidine, tilisolol, timolol, tinidazole, tinofedrine, tinzaparin, tioconazole, tipranavir, tirapazamine, tirofiban, tiropramide, titanicene, tizanadine, tocainide, tolazamide, tolazoline, tolbutamide, tolcapone, tolciclate, tolfenamic acid, toliprolol, tolteridine, tolterodine, tonaberstat, topiramate, topotecan, torasemide, toremifene citrate, toremifene, tosufloxacin, tramadol, tramazoline, trandolapril, tranilast, tranylcypromine, trapidil, traxanox, trazodone, tretoquinol, triacetin, triamcinolone, triampterine, triamterine, triazolam, triazoles, tricromyl, tricyclics, trifluoperazine hydrochloride, trifluoperazine, triflupromazine, trifluridine, trihexyphenidyl hydrochloride, trihexyphenidyl, trimazosin, trimebutine, trimetazidine, trimethoprim, trimegestone, trimipramine, trimoprostil, trithiozine, troglitazone, trolnitrate phosphate, tromethamine, tropicamide, trovafloxacin, troxipide, triaminoheptane, tulobuterol, tymazoline, tyramine, undecanoate, undecanoic acid, urinastatin, ursodeoxycholic acid, valacyclovir, valdecoxib, valerate, valganciclovir, valproic acid, valsartan, vancomycin, vardenafil, venlafaxine, venorelbine, verapamil, vidarabine, vigabatrin, vincamine, vinpocetine, viomycin, viquidil, visnadine, vitamin A derivatives, vitamin A, vitamin B2, vitamin D, vitamin E, vitamin K, voglibose, voriconazole, xaliproden, xamoterol, xanthinol niacinate, xenytropium bromide, xibenolol, ximelagatran, xylometazoline, yohimbine, zacopride, zafirlukast, zalcitabine, zaleplon, zanamivir, zatebradine, ziconotide, zidovudine, zileuton, zimeldine, zinc propionate, ziprasidone, zolimidine, zolnitriptan, zolpidem, zonisamide, and zopiclone.

C. Optional Components

If desired, the pharmaceutical compositions of the present invention can optionally include additional compounds to enhance the solubility of the therapeutic agent or the triglyceride in the composition. It must be emphasized, however, that while such solubilizers may be beneficial in certain compositions, they are not required in the present compositions to achieve the advantages discussed herein, e.g., an increase in the rate and/or extent of absorption. Examples of solubilizers include, but are not limited to, the following:

alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulosic polymers, cyclodextrins and cyclodextrin derivatives;

ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide);

amides, such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, and polyvinylpyrrolidone;

esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof;

and other solubilizers known in the art, such as dimethyl acetamide, dimethyl isosorbide (Arlasolve DMI (ICI)), N-methylpyrrolidones (Pharmasolve (ISP)), monooctanoin, diethylene glycol monoethyl ether (available from Gattefosse under the trade name Transcutol), and water.

Mixtures of solubilizers are also within the scope of the invention. Except as indicated, these compounds are readily available from standard commercial sources.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included in compositions of the present invention is not particularly limited. Of course, when the composition is ultimately administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of therapeutic agent, with excess solubilizer removed prior to providing the composition to a patient using conventional techniques, such as distillation or evaporation. Thus, if present, the solubilizer can be in a concentration of 50%, 100%, 200%, or up to about 400% by weight, based on the amount of surfactant. If desired, very small amounts of solubilizers may also be used, such as 25%, 10%, 5%, 1% or even less. Typically, the solubilizer will be present in an amount of about 1% to about 100%, more typically about 5% to about 25% by weight or about 10% to about 25% by weight.

Other additives conventionally used in pharmaceutical compositions can also be included, such as, for example, detackifiers, anti-foaming agents, buffering agents, antioxidants, preservatives, chelating agents, viscomodulators, tonicifiers, flavorants, colorants odorants, opacifiers, suspending agents, binders, fillers, plasticizers, lubricants, and mixtures thereof. Preferred additives are antioxidants, viscomodulators, and suspending agents. The amounts of any of these optional additives can be readily determined by one skilled in the art, according to the particular properties desired. Of course, it will be appreciated that the presence of one or more additives in the compositions, and in the ultimate dosage form, may result in a dispersion that is not clear, i.e., a dispersion having an absorbance of greater than about 0.3 at 400 nm.

D. Concentrations

The components of the present compositions are present in amounts such that upon dilution with an aqueous medium, the composition forms a clear, aqueous dispersion. The determining concentrations of components to form clear aqueous dispersions are the concentrations of triglyceride and surfactants, with the amount of the therapeutic agent being selected as described below. The relative amounts of triglycerides and surfactants are readily determined by observing the properties of the resultant dispersion; i.e., when the relative amounts of these components are within a suitable range, the resultant aqueous dispersion is optically clear. When the relative amounts are outside the suitable range, the resulting dispersion is visibly “cloudy,” resembling a conventional emulsion or multiple-phase system. Although a visibly cloudy solution may be potentially useful for some applications, such a system would suffer from many of the same disadvantages as conventional prior art compositions, as described above.

The aqueous medium can comprise body fluids naturally occurring in the subject to whom the pharmaceutical compositions are administered. Such naturally occurring fluids can be the fluids occurring or produced in the oral cavity, nasal cavity, respiratory system, digestive system, for example, gastric juice, intestinal fluid, saliva, and lung fluid. The aqueous medium can also be fluids simulating such naturally occurring body fluids, for example, simulated gastric fluid and simulated intestinal fluid in absence or presence of variable amount of naturally occurring, semi-synthetic, or synthetic surface active materials. Typical surface active materials include proteins such as pepsin and pancreatin (which also possess enzymatic activity), bile acids, bile salts, phospholipids such as lecithins and lysolecithins and synthetic surfactant such as Tweens, sodium lauryl sulfate, etc. The concentration of such materials present in the simulated fluids can be in the range of about 0.01 wt. % to about 10 wt. %, most typically in the range of about 0.01 wt. % to about 1 wt. %. Occasionally, other organic materials such glycerol, alcohol, and polymers such as PEG and PVP can be incorporated in the simulated fluids to adjust properties such as viscosity, osmolarity, and dielectric constant; such materials can also serve as solubilizing agents.

A convenient method of determining the appropriate relative concentrations for any particular triglyceride is as follows. A convenient working amount of a hydrophilic surfactant is provided, and a known amount of the triglyceride is added. The mixture is stirred, with the aid of gentle heating if desired, then is diluted with purified water to prepare an aqueous dispersion. Any dilution amount can be chosen, but convenient dilutions are those within the range expected in vivo, about a 10 to 250-fold dilution. The aqueous dispersion is then assessed qualitatively for optical clarity. The procedure can be repeated with incremental variations in the relative amount of triglyceride added, to determine the maximum relative amount of triglyceride that can be present to form a clear aqueous dispersion with a given hydrophilic surfactant, i.e., when the relative amount of triglyceride is too great, a hazy or cloudy dispersion is formed.

The amount of triglyceride that can be solubilized in a clear aqueous dispersion is increased by repeating the above procedure, but substituting a second hydrophilic surfactant, or a hydrophobic surfactant, for part of the originally-used hydrophilic surfactant, thus keeping the total surfactant concentration constant. Of course, this procedure is merely exemplary, and the amounts of the components can be chosen using other methods, as desired.

It has been surprisingly found that mixtures of surfactants including two surfactants can solubilize a greater relative amount of triglyceride than a single surfactant. Similarly, mixtures of surfactants including a hydrophilic surfactant and a hydrophobic surfactant can solubilize a greater relative amount of triglyceride than either surfactant by itself. It is particularly surprising that when the surfactant mixture includes a hydrophilic surfactant and a hydrophobic surfactant, the solubility of the triglyceride is greater than, for example, in the hydrophilic surfactant itself. Furthermore, a greater amount of the hydrophobic surfactant can be solubilized when a triglyceride is present for a given amount of a hydrophilic surfactant. Thus, contrary to conventional knowledge in the art, the total amount of water-insoluble component (triglyceride plus hydrophobic surfactant) exceeds the amount of hydrophobic surfactant or triglyceride that can be solubilized by the same amount of hydrophilic surfactant. This unexpected finding shows a surprising and non-intuitive relationship between the hydrophilic and hydrophobic components.

It should be emphasized that the optical clarity is determined in the diluted composition (the aqueous dispersion), and not in the pre-concentrate. Thus, for example, U.S. Pat. No. 4,719,239 shows optically clear compositions containing water, oil, and a 3:7 mixture of PEG-glycerol monooleate and caprylic-capric acid glycerol esters, but the compositions contain no more that about 75% by weight water, or a dilution of the pre-concentrate of no more than 3 to 1. Upon dilution with water in a ratio of more than about 3 to 1, the compositions of the cited reference phase-separate into multi-phase systems, as is shown, for example, in the phase diagram of FIG. 2 in the '239 patent. In contrast, the compositions of the present invention, when diluted to values typical of dilutions encountered in vivo, or when diluted in vivo upon administration to a patient, remain as clear aqueous dispersions. Thus, the clear aqueous dispersions of the present invention are formed upon dilution of about 10 to about 250-fold or more.

In the compositions of the invention, then, the triglyceride and surfactants should be present in amounts that are pharmaceutically acceptable and selected so that upon admixture of the composition with an aqueous medium in an aqueous medium to composition ratio of about 250:1, preferably 100:1, and most preferably 10:1 by weight, a clear aqueous dispersion is formed, generally exhibiting an absorbance of less than about 0.3 at 400 nm. Preferred compositions are those wherein the clear aqueous dispersion formed exhibits an absorbance of less than about 0.2 at 400 nm, with particularly preferred compositions resulting in a clear aqueous dispersion exhibiting an absorbance of less than 0.1 at 400 nm. The aforementioned parameters hold true whether or not the active agent is incorporated into the compositions (see Example 7).

As an alternative to qualitative visual assessment of optical clarity, the optical clarity of the aqueous dispersion can be measured using standard quantitative techniques for turbidity assessment. One convenient procedure to measure turbidity is to measure the amount of light of a given wavelength transmitted by the solution, using, for example, a UV-visible spectrophotometer. Using this measure, optical clarity corresponds to high transmittance, since cloudier solutions will scatter more of the incident radiation, resulting in lower transmittance measurements. If this procedure is used, care should be taken to insure that the composition itself does not absorb light of the chosen wavelength, as any true absorbance necessarily reduces the amount of transmitted light and falsely increases the quantitative turbidity value. Analogously, any water-insoluble solid particles present in the composition (except for precipitated or undissolved drug particles formed as a result of dispersing the composition in an aqueous medium) must be discounted from the absorbance of the aqueous dispersion in determining the clarity thereof In the absence of chromophores at the chosen wavelength, suitable dispersions at a dilution of 100× should have an apparent absorbance of less than about 0.3, preferably less than about 0.2, and more preferably less than about 0.1, and when the term “absorbance” is used herein, it is intended to refer to the apparent absorbance corrected (normalized) with respect to the presence of water-insoluble solid particles as described above.

It should be emphasized that any or all of the available methods may be used to ensure that the resulting aqueous dispersions possess the requisite optical clarity. For convenience, however, the present inventors prefer to use the simple qualitative procedure; i.e., simple visible observation. However, in order to more fully illustrate the practice of the present invention, both qualitative observation and spectroscopic measures are used to assess the dispersion clarity in the Examples herein.

In one embodiment, the invention provides an orally administrable composition wherein a therapeutic agent, e.g., a lipid-regulating agent, is present in an amount up to the maximum that can be solubilized in the triglyceride, the surfactants, or both the triglyceride and the surfactants.

In another embodiment, a therapeutic agent, e.g., a lipid-regulating agent, is present in a first amount that is solubilized, and a second amount that remains unsolubilized but dispersed. This may be desirable when, for example, a larger dose of the active agent is desired. Although not all of the active agent is solubilized, such a composition presents advantages over conventional compositions, since at least a portion of the active agent is present in the clear aqueous dispersion phase. Of course, in this embodiment, the optical clarity of the resultant aqueous dispersion is determined before the second non-solubilized amount of the therapeutic agent is added.

Alternatively, the active agent can be solubilized in the aqueous medium used to dilute the preconcentrate to form an aqueous dispersion. The maximum amount of active agent that can be solubilized is readily determined by simple mixing, as the presence of any non-solubilized agent is apparent upon visual examination. As emphasized elsewhere herein, in all of the embodiments described herein, the triglyceride and surfactants are present in amounts such that upon admixture of the composition with an aqueous medium, either in vitro or in vivo, a clear, aqueous dispersion is formed. This optical clarity in an aqueous dispersion defines the appropriate relative concentrations of the triglyceride and surfactant components, but does not restrict the dosage form of the compositions to an aqueous dispersion, nor does it limit the compositions of the invention to optically clear dosage forms. Thus, the appropriate concentrations of the triglyceride and surfactants are determined by the optical clarity of a dispersion formed by the composition preconcentrate and an aqueous medium in a dilution of about 10 to about 250-fold, as a preliminary matter.

Once the appropriate concentrations are determined, the pharmaceutical compositions can be formulated as described in the preceding section, without regard to the optical clarity of the ultimate composition. Of course, optically clear aqueous dispersions, and their preconcentrates, are preferred compositions.

In some contexts, the compositions will be “substantially free of water.” “Substantially free of water” as used herein is intended to mean that the composition or dosage form contains less than 20% water (v/v). More preferably, the composition or dosage form contains less than about 10% water and most preferably less than about 5% water. In turn, this means that any water present will not form a continuous aqueous phase.

Other considerations well known to those skilled in the art will further inform the choice of specific proportions of components, e.g., surfactants and triglycerides, of the compositions. These considerations include the degree of bioacceptability of the compounds, and the desired dosage of therapeutic agent to be provided. In some cases, the amount of triglyceride or therapeutic agent actually used in a pharmaceutical composition according to the present invention will be less than the maximum that can be solubilized, and it should be apparent that such compositions are also within the scope of the present invention.

E. Preferred Compositions:

Preferred compositions of the invention are those that provide a significantly improved rate and/or extent of absorption of drug relative to an analogous composition or a conventional formulation/dosage form that do not meet the clarity requirements herein. Typically, the analogous composition comprises (a) the drug, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight. The conventional formulation or dosage form can be a tablet or a capsule encapsulating powders or pellets comprising the drug. That is, following oral administration under an identical dosage regimen (which, again, refers to a dosage regimen that is identical not only with respect to drug dose, but also with respect to meal timing and meal content, particularly meal fat content), the composition provides an increase in the rate of absorption of the drug relative to the rate of absorption from the corresponding composition after dose normalization. As explained elsewhere herein, dose normalization is necessary when the dose of the drug in the composition of the present invention is different from that in the corresponding composition for the comparison of the extent of drug absorption.

The increase in the rate of absorption corresponds to and may be determined by the amount of time required to reach maximum plasma concentration of the drug or an active metabolite thereof. With the present compositions, the increase in the rate of absorption is such that the time to reach maximum plasma concentration of the drug or an active metabolite thereof is preferably reduced by at least 10%, more preferably by at least 25%, and most preferably by at least 50%.

Preferred therapeutic agents herein are lipid-regulating agents, as noted elsewhere herein. When the lipid-regulating agent is fenofibrate, the increase in the rate of absorption corresponds to and may be determined by the amount of time required to reach maximum plasma concentration of fenofibric acid, the active metabolite of fenofibrate. When comparing to a conventional formulation/dosage form, for example, the Tricor® (Abbott) fenofibrate, a hard gelatin capsule composed of fenofibrate and other inactive ingredients including lactose, sodium lauryl sulfate, crospovidone, magnesium stearate and pregelatinized starch in powder form, wherein said fenofibrate is co-micronized with sodium lauryl sulfate, the composition of the present invention provides an increase in the rate of absorption of fenofibrate over the Tricor capsule. With the present compositions, the increase in the rate of fenofibrate absorption is such that the time to reach maximum plasma concentration of fenofibric acid is preferably reduced by at least 20%, more preferably by at least 50%, and most preferably by at least 75% over the Tricor capsule.

In a preferred embodiment, the invention provides compositions for the administration of lipid-regulating agents, particularly fibric acid derivatives such as bezafibrate, beclobrate, binifibrate, ciprofibrate, clinofibrate, clofibrate, etofibrate, fenofibrate, gemfibrozil, nicofibrate, pirifibrate, ronifibrate, simfibrate, and theofibrate (or their corresponding acid forms), with fenofibrate most preferred.

Particularly preferred compositions herein, including, but not limited to, fenofibrate compositions, do not contain any components that are not “pharmaceutically acceptable,” nor are any components present in excess of pharmaceutically acceptable levels. Such compositions exclude, by way of example, propylene glycol fatty acid esters.

With respect to the increased extent of absorption, the present compositions in fact give rise to an increase in the extent of absorption of the drug relative to the extent of absorption for a corresponding composition, administered under an identical dosage regimen, containing (a) the drug, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization. The increase in the extent of absorption may be determined by the area under the curve (AUC) of the plasma concentration of the drug or an active metabolite thereof as a function of time. With the present compositions, the increase in the extent of absorption provided by the invention is such that the AUC of the plasma concentration of the drug or an active metabolite thereof is preferably increased by at least 10%, more preferably by at least 20%, and most preferably by at least 50%.

As noted above, in a more preferred embodiment, the drug is a lipid-regulating agent. When the lipid-regulating agent is fenofibrate, the increase in the extent of absorption corresponds to and may be determined by the AUC of the plasma concentration of fenofibric acid. When compared to a conventional formulation/dosage form, for example, the Tricor® (Abbott) fenofibrate, a hard gelatin capsule composed of fenofibrate and other inactive ingredients including lactose, sodium lauryl sulfate, crospovidone, magnesium stearate and pregelatinized starch in dry powder form, wherein said fenofibrate is co-micronized with sodium lauryl sulfate, the composition of the present invention provides an increase in the extent of absorption of fenofibrate over the Tricor capsule after dose normalization. With the present compositions, the increase in the extent of absorption provided by the invention is such that the AUC of the plasma concentration of fenofibric acid is preferably increased by at least 20%, more preferably by at least 50%, and most preferably by at least 75% after dose normalization. In general, the fenofibrate compositions of the invention provide an area under the curve from time zero to infinity (AUC_{0-inf}) of the plasma concentration of fenofibric acid as a function of time, per milligram of dosed fenofibrate, of at least 1.0 μg·hr/ml and more preferably, at least 1.25 μg·hr/ml, following oral administration of the composition to a human patient.

Additionally, the preferred compositions of the invention provide for less dependency of drug absorption on lipolysis and endogenous bile, bile-related patient disease states, or meal fat contents, relative to a conventional formulation/dosage form or an analogous composition containing (a) the drug, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization. “Less dependency” of a therapeutic agent (e.g., fenofibrate) on lipolysis and endogenous bile, bile-related patient disease states, or meal fat contents can be defined as the extent of absorption The term “less dependency” with respect to the absorption of drugs, for example, lipid-regulating agents, particularly fenofibrate, is evidenced by an extent of absorption (expressed as AUC of the drug or active metabolite(s) of the drug) of the drug within 67-150% and more preferably, within 80-125%, for the composition when administered under any two different meal conditions. Meal fat content can be defined as high-fat, low-fat and non-fat. The category of “non-fat” includes a non-fat meal or no meal (fasting). In a high-fat meal, approximately 50% or more of total caloric content of the meal is derived from the fat. An example of a high-fat meal is illustrated in the “guidance for industry: food-effect bioavailability and fed bioequivalences studies” posted by FDA in December 2002. An example of a test high-fat and high-calorie meal from the aforementioned report is a meal of two eggs fried in butter, two strips of bacon, two slices of toast with butter, four oz. of hash brown potatoes and eight oz. of whole milk, which derives approximately 150, 250 and 500-600 calories from protein, carbohydrate and fat, respectively. In a low-fat meal, approximately 25-35% of total caloric content of the meal is derived from the fat. Examples of such low-fat meal can be found in the therapeutic lifestyle changes (TLC) diet provided in the adult treatment panel III (ATP III) report by national cholesterol education program (NCEP). Typically, APT III allows total fat to 35% of total calories including up to 10% and 20% of total calories from polyunsaturated fat and monounsaturated fat, respectively. In a non-fat meal, less than approximately 10% of total caloric content of the meal is derived from the fat. For example, a light breakfast will consist of 6 oz. cereal with 8 oz. of fat-free milk or 2% fat milk, 6 oz. orange juice, two slices of toast (jelly allowed), a 4 oz. portion of fruit and 1 cup of coffee (only sugar allowed).

To compare different formulations/dosage forms, the timing of the meal with respect to the administration of different formulations/dosage forms should be identical, e.g., using the aforementioned FDA report on evaluating food-effect bioavailability and fed bioequivalences. Typically, under fasting conditions, following an overnight fast of at least 10 hours, subjects should be administered the drug product with 240 ml of water. No food should be allowed for at least 4 hours post-dose. Water can be allowed as desired except for one hour before and after drug administration. Under fed condition, following an overnight fast of at least 10 hours, subjects should start the meal 30 minutes prior to administration of the drug product. Study subjects should eat the meal in 30 minutes or less; however, the drug product should be administered 30 minutes after start of the meal.

In the more preferred compositions herein:

(a) the triglyceride component is a medium chain triglyceride, preferably in the form of a triglyceride containing predominantly C₆-C₁₂ fatty acids such as glyceryl tricaprylate/caprate, as may be obtained under the tradenames Captex 300 (Abitec), Captex 355 (Abitec), Miglyol 810 (Hüls), and Miglyol 812 (Hüls);

(b) the at least one hydrophilic surfactant is selected from polyoxyethylene sorbitan fatty acid esters, polyoxyethylene vegetable oil, polyoxyethylene hydrogenated vegetable oil, and hydrophilic transesterification products of oils (including oil-soluble vitamins) and alcohol and mixtures thereof, with polysorbate 80, PEG-35 castor oil, PEG-40 castor oil, PEG-8 caprylic/capric glycerides, lauroyl macrogol-32 glycerides, stearoyl macrogol glyceride, and tocopheryl PEG-1000 succinate particularly preferred. In these compositions, the carrier may also include at least one hydrophobic surfactant, e.g., a surfactant selected from hydrophobic transesterification products of an oil (including an oil-soluble vitamin) and an alcohol, and glycerol fatty acid esters such as monoglycerides, diglycerides, and mixtures thereof, with particularly preferred hydrophobic surfactants selected from transesterification products of PEG-6 corn oil, PEG-6 apricot kernel oil, and mixtures thereof, glycerol fatty acid esters selected from monoglycerides, diglycerides, and mixtures thereof, with preferred glycerol fatty acid esters being glyceryl caprylate, glyceryl caprylate/caprate and mixtures thereof, as well as glyceryl linoleate, glyceryl monooleate, glyceryl dioleate and mixtures thereof, and propylene glycol fatty acid esters, with preferred such esters being propylene glycol monocaprylate, propylene glycol dicaprylate/dicaprate, and mixtures thereof. In optimal compositions, however, as noted above, propylene glycol fatty acid esters are not included, insofar as at least several such surfactants are not “pharmaceutically acceptable” as defined herein.

Accordingly, in a particularly preferred embodiment, the invention provides a composition that comprises:

(a) a carrier comprising glyceryl tricaprylate/caprate, at one hydrophilic surfactant selected from the group consisting of tocopheryl PEG-1000 succinate, polysorbate 80, PEG-35 castor oil, PEG-40 hydrogenated castor oil and mixtures thereof; and at least one hydrophobic surfactant selected from the group consisting of glyceryl caprylate, glyceryl caprylate/caprate, mixtures thereof; and

(b) a therapeutically effective amount of fenofibrate,

wherein the triglyceride and surfactants are present in amounts that are pharmaceutically acceptable and selected so that upon admixture of the composition with an aqueous solution in an aqueous solution to composition ratio of about 100:1 by weight, a clear aqueous dispersion having an absorbance of less than about 0.3 at 400 nm is provided. In this composition, the at least one hydrophilic surfactant is optimally selected from tocopheryl PEG-1000 succinate, polysorbate 80, and mixtures thereof.

The aforementioned fenofibrate composition, following oral administration, provides an increase in the rate and/or an increase in the extent of absorption of fenofibrate compared to a corresponding composition in the form of a capsule containing fenofibrate co-micronized with a solid surfactant, particularly sodium lauryl sulfate, after dose normalization. The aforementioned capsule corresponds to the Tricor® (Abbott) fenofibrate product, a hard gelatin capsule composed of fenofibrate and other inactive ingredients including lactose, sodium lauryl sulfate, crospovidone, magnesium stearate and pregelatinized starch in dry powder form, wherein said fenofibrate is co-micronized with sodium lauryl sulfate. See also U.S. Pat. No. 4,895,726 to Curtet et al. and the monograph of Tricor® (fenofibrate capsule) wherein the capsule is that available.

F. Multi-Phase Dispersions:

The composition may also be a multi-phase dispersion containing the therapeutic agent. In this embodiment, the composition results in a clear aqueous dispersion upon dilution with an aqueous medium as explained elsewhere herein, and further includes an additional amount of non-solubilized active agent. Thus, the term “multi-phase” as used herein to describe these compositions of the present invention means a composition which when mixed with an aqueous medium forms a clear aqueous phase and a particulate dispersion phase. The carrier components are as described above, and can include any of the surfactants, therapeutic agents, solubilizers, and additives previously described. An additional amount of the therapeutic agent is included in the composition. This additional amount is not solubilized by the carrier, and upon mixing with an aqueous system is present as a separate dispersion phase. The additional amount is optionally a milled, micronized, or precipitated form. Thus, upon dilution, a two-phase system is formed: a clear aqueous dispersion of the triglyceride and surfactants containing a first, solubilized amount of the therapeutic agent, and a second, non-solubilized amount of the agent dispersed therein. It should be emphasized that the resultant multi-phase dispersion will not have the optical clarity of a dispersion in which the active agent is fully solubilized, but will appear to be cloudy, due to the presence of the non-solubilized phase. Such a composition may be useful, for example, when the desired dosage of an active agent exceeds that which can be solubilized in the carrier and/or triglyceride.

One skilled in the art will appreciate that a particular therapeutic agent may have a greater solubility in the pre-concentrate composition than in the aqueous dispersion, so that meta-stable, supersaturated solutions having apparent optical clarity but containing an active agent in an amount in excess of its solubility in the aqueous dispersion can be formed. Such super-saturated solutions, whether characterized as clear aqueous dispersions (as initially formed) or as multi-phase solutions (as would be expected if the meta-stable state breaks down), are also within the scope of the present invention.

The multi-phase composition can be prepared by the methods described above. A pre-concentrate is prepared by simple mixing of the components, with the aid of gentle heating, if desired. It is convenient to consider the active agent as divided into two portions, a first solubilizable portion that will be solubilized and contained within the clear aqueous dispersion upon dilution, and a second non-solubilizable portion that will remain non-solubilized. When the ultimate dosage form is non-aqueous, the first and second portions of the agent are both included in the pre-concentrate mixture. When the ultimate dosage form is aqueous, the composition can be prepared in the same manner, and upon dilution in an aqueous system, the composition will form the two phases as described above, with the second non-solubilizable portion of the active agent dispersed or suspended in the aqueous system, and the first solubilizable portion of the active agent solubilized in the composition. Alternatively, when the ultimate dosage form is aqueous, the pre-concentrate can be prepared including only the first, solubilizable portion of the active agent. This pre-concentrate can then be diluted in an aqueous system to form a clear aqueous dispersion, to which is then added the second, non-solubilizable portion of the active agent to form a multi-phase aqueous composition.

III. Dosage Forms, Preparation, and Methods of Use:

The pharmaceutical compositions of the present invention can be formulated as a preconcentrate in a liquid, semi-solid, or solid form, or as an aqueous or organic diluted preconcentrate. In the diluted form, the diluent can be water, an aqueous medium, a buffer, an organic solvent, a beverage, a juice, or mixtures thereof. If desired, the diluent can include components soluble therein, such as solubilizers and other optional additives.

The composition form is orally administrable, such that all components and amounts thereof are pharmaceutically acceptable for an oral dosage form. The dosage form itself is not particularly limited, and compositions of the present invention can be formulated as pills, capsules, caplets, tablets, granules, beads or powders. Granules, beads and powders can, of course, be further processed to form pills, capsules, caplets or tablets. When formulated as a capsule, the capsule can be a hard or soft gelatin capsule, a starch capsule, or a cellulosic capsule. Such dosage forms can further be coated with, for example, a seal coating or an enteric coating. Use of a solubilizer is particularly preferred in capsule dosage forms of the present compositions. If present, these solubilizers should be added in amounts sufficient to impart to the compositions the desired solubility enhancement or encapsulation properties.

The pharmaceutical compositions of the present invention can be prepared by conventional methods, e.g., lyophilization, encapsulation, compression, melting, extrusion, drying, chilling, molding, spraying, coating, comminution, mixing, homogenization, sonication and granulation. Of course, the specific method of preparation will depend upon the ultimate dosage form. For dosage forms substantially free of water, i.e., when the composition is provided in a pre-concentrate form for later dispersion in vitro or in vivo in an aqueous system, the composition is prepared by simple mixing of the components to form a pre-concentrate. The mixing process can be aided by gentle heating, if desired. For compositions in the form of an aqueous dispersion, the pre-concentrate form is prepared, and the appropriate amount of an aqueous medium is then added. Upon gentle mixing, a clear aqueous dispersion is formed. If any water-soluble additives are included, these may be added first as part of the pre-concentrate, or added later to the clear aqueous dispersion, as desired.

In another embodiment, the present invention relates to a method of increasing the rate and/or extent of absorption of a therapeutic agent by administering a pharmaceutical composition of the invention to a patient. In this embodiment, the therapeutic agent can be present in the pharmaceutical composition pre-concentrate, in the diluent, or in a second pharmaceutical composition, such as a conventional commercial composition, which is co-administered with a pharmaceutical composition of the present invention. For example, the delivery of therapeutic agents in conventional pharmaceutical compositions can be improved by co-administering a pharmaceutical composition of the present invention with a conventional composition.

Administration of the compositions and dosage forms described herein may be used to treat any disorder for which a particular therapeutic agent may be indicated, e.g., any lipid disorder for which a particular lipid-regulating agent is generally indicated. In the latter case, the lipid disorder may be a metabolic disorder, as may be associated with a metabolic syndrome. Specific lipid disorders include, by way of example, hypercholesterolemia, mixed dyslipidemia, and hypertriglyceridemia.

Dosage regimens and daily dosage for the lipid-regulating agent can vary a great deal, as a number of factors are involved, including the particular active agent, the age and general condition of the patient, the particular condition or disorder and its severity, and the like. Clearly, however, it is necessary that the dosage given be sufficient to provide the desired pharmacological activity in a patient's circulation. In general, suitable dosages can be determined by one of ordinary skill in the art without undue experimentation. For example, suitable dosages for humans can be determined based on data derived from animal studies. In addition, suitable dosages may be determined by administering an estimated dose, noting the response, and adjusting the dose accordingly. The daily dosage of fenofibrate, in particular, will generally be in the range of about 50-200 mg/day, with typical unit dosages of about 40-60 mg, e.g., 54 mg, or of about 160-180 mg, e.g., 168 mg. A higher dosage is generally required for treatment of hypercholesterolemia and mixed dyslipidemia, while a lower dosage is normally sufficient, at least initially, in the treatment of hypertriglyceridemia. However, the actual dose of the drug could be adjusted depending on the particular indication, the particular patient's response to the drug and/or dosage thereof, and the enhanced absorption provided by the compositions of the present invention. With regard to the latter consideration, the effective dose (i.e., the minimum therapeutically effective dose) can generally be reduced relative to the necessary dose required with currently available commercial drug products and/or with compositions that are analogous to those herein but which do not meet the clarity requirement.

In a related embodiment, the invention provides a method for reducing the dependency of absorption on lipolysis of an orally administered lipid-regulating agent, e.g., fenofibrate, by administering the agent in a composition that comprises a carrier that includes a triglyceride and at least two surfactants, at least one of which is hydrophilic, and a therapeutically effective amount of the lipid-regulating agent, wherein the triglyceride and surfactants are present in amounts that are pharmaceutically acceptable and are selected so that so that upon admixture of the composition with an aqueous medium in an aqueous medium to composition ratio of about 100:1 by weight, a clear aqueous dispersion having an absorbance of less than about 0.3 at 400 nm is provided.

Analogously, the invention provides a method for reducing the dependency of absorption on endogenous bile, bile-related patient disease states, or meal fat contents of an orally administered lipid-regulating agent, e.g., fenofibrate, by administering the agent in a composition that comprises a carrier that includes a triglyceride and at least two surfactants, at least one of which is hydrophilic, and a therapeutically effective amount of the lipid-regulating agent, wherein the triglyceride and surfactants are present in amounts that are pharmaceutically acceptable and are selected so that so that upon admixture of the composition with an aqueous medium in an aqueous medium to composition ratio of about 100:1 by weight, a clear aqueous dispersion having an absorbance of less than about 0.3 at 400 nm is provided.

The pharmaceutical compositions or the aqueous dispersions formed upon dilution of the compositions described herein will generally have the following characteristics:

Rapid formation: upon dilution with an aqueous medium, the composition provides a clear dispersion very rapidly; i.e., the clear dispersion appears to form instantaneously.

Optical clarity: the dispersions are essentially optically clear to the naked eye, and show no readily observable signs of heterogeneity, such as turbidity or cloudiness. More quantitatively, dispersions of the carrier compositions of the present invention generally, although not necessarily, have an absorbance at 400 nm less than about 0.3, preferably less than about 0.2, and often less than about 0.1, at 100× dilution, as described more fully elsewhere herein. In the multi-phase embodiment described herein, however, it should be appreciated that the optical clarity of the aqueous phase will be obscured by the dispersed particulate non-solubilized therapeutic agent.

Robustness to dilution: the dispersions are surprisingly stable to dilution in aqueous medium. The hydrophobic therapeutic agent remains solubilized for at least the period of time relevant for absorption.

As discussed above, conventional triglyceride-containing compositions suffer from the disadvantage that bioabsorption of therapeutic agents contained therein is dependent upon enzymatic degradation (lipolysis) of the triglyceride components of the composition. The solubilization of the triglyceride in an aqueous medium is normally limited when only a hydrophilic surfactant is used to disperse the triglyceride, as is conventionally the case. Without a sufficiently high concentration of the hydrophilic surfactant, an emulsion or milky suspension of the triglyceride is formed, and the triglyceride is present in the form of relatively large oil droplets, which can, in turn, impede the transport and absorption of the triglyceride or therapeutic agent solubilized in the triglyceride or in the carrier. In addition, the large, thermodynamically unstable triglyceride particles could further impose a risk when the compositions are administered intravenously, by plugging the blood capillaries.

To achieve a high level of fully solubilized triglyceride would require a substantial amount of the hydrophilic surfactant, far exceeding that which would be bioacceptable. The pharmaceutical compositions of the present invention, however, by virtue of containing a third component, either a hydrophobic surfactant or a second hydrophilic surfactant. The solubilization of the triglyceride in the aqueous system is thereby enhanced. Conversely, it is also true that solubilization of a hydrophobic surfactant or a second hydrophilic surfactant is enhanced based on the presence of the triglyceride in the composition. Of course, the relative amounts of the hydrophobic surfactant and/or a second hydrophilic surfactant in the composition will depend on the type of composition, the actual components used, the nature of the therapeutic agent, and so forth. These and other factors are routinely considered by those of skill in the art in determining the optimal amount of each component in a composition.

It has also been found that the total amount of solubilized water-insoluble components, i.e., the triglyceride and the hydrophobic surfactant, can greatly exceed the amount of the triglyceride or hydrophobic surfactant alone that would be solubilized using the same amount of the hydrophilic surfactant.

In addition to forming a thermodynamically stable aqueous dispersion upon admixture with an aqueous medium, the present compositions may also form optically clear, meta-stable or supersaturated dispersions with respect to the therapeutic agent and/or the triglyceride/hydrophobic surfactant in an amount in excess of the equilibrium solubility of the aqueous dispersion. Super-saturated solutions, whether characterized as homogeneous, single-phase, and clear aqueous dispersions (as initially formed), or as multi-phase solutions (as would be expected if the meta-stable state breaks down), are also within the scope of the present invention. It is particularly desirable, however, that a meta-stable or supersaturated composition containing the therapeutic agent, triglyceride, and/or the hydrophobic surfactant is formed in the aqueous dispersion for at least a period of time sufficient for the absorption of the therapeutic agent in vivo. A suitable time period will be known by one of ordinary skill in the art. Generally, up to about eight hours, more typically from about one to about four hours, upon forming the dispersion is a sufficient time period for absorption of the therapeutic agent in vivo.

The unique pharmaceutical compositions and methods of the present invention accordingly provide for a number of significant advantages, including, but not limited to, the following.

Efficient transport: The increased degree of drug solubilization in the compositions of the present invention enables more efficient drug transport through the intestinal aqueous boundary layer, and through the absorptive brush border membrane. More efficient transport to absorptive sites leads to improved and more consistent absorption of therapeutic agents.

Less dependence on lipolysis: The present pharmaceutical compositions less dependent upon lipolysis and upon the many poorly characterized factors that affect the rate and extent of lipolysis, for effective presentation of a therapeutic agent to an absorptive site. Such factors include the presence of components that may inhibit lipolysis; patient conditions which limit production of lipase, such as pancreatic lipase secretory diseases; and dependence of lipolysis on stomach pH, endogenous calcium concentration, and presence of co-lipase or other digestion enzymes. The reduced lipolysis dependence further provides transport that is less prone to suffer from any lag time between administration and absorption caused by the lipolysis process, enabling a more rapid onset of therapeutic action and better bioperformance characteristics. In addition, pharmaceutical compositions of the present invention can make use of hydrophilic surfactants that might otherwise be avoided or limited due to their potential lipolysis inhibiting effects.

Non-dependence on bile and meal fat contents: Due to the higher solubilization potential over bile salt micelles, the present compositions are less dependent on endogenous bile and bile related patient disease states, and meal fat contents. These advantages overcome meal-dependent absorption problems caused by poor patient compliance with meal-dosage restrictions.

Superior solubilization: The triglyceride and surfactant combinations used in compositions of the present invention enable superior loading capacity over conventional compositions. Thus, for example, more therapeutic agent can be solubilized in the triglyceride and surfactant combinations described herein than would be possible with conventional compositions containing only surfactant alone. Stated differently, the presence of the triglyceride in the present combinations improves the loading of the therapeutic agent for any given surfactant level. In addition, the particular combination of surfactants used can be optimized for a specific therapeutic agent to more closely match the polarity distribution of the therapeutic agent, resulting in still further enhanced solubilization.

Superior loading/presentation of absorption enhancers: The triglyceride and surfactant combinations in the present compositions enhance the compositions' loading capacity with respect to absorption enhancers incorporated therein, and also provide for superior presentation of the enhancers at the absorption sites, relative to conventional compositions. Consequently, the invention also includes a method for increasing the loading capacity of a pharmaceutical composition by providing: a pharmaceutical composition comprised of (a) a carrier comprising a triglyceride and a first surfactant, and (b) a therapeutically effective amount of a therapeutic agent; and adding an absorption-enhancing amount of a second surfactant to the pharmaceutical composition, the second surfactant comprising a hydrophobic surfactant, wherein the absorption-enhancing amount is effective to increase the loading capacity of the composition. Preferred absorption enhancers include, without limitation, those mentioned in the overviews provided by Muranishi (1990), “Absorption Enhancers,” Critical Reviews in Therapeutic Drug Carrier Systems 7 (1):1-33; Aungst (2000), “Intestinal Permeation Enhancers,” J. Pharm. Sci. 89(4):429-442 and Curatolo et al. “Safety Assessment of Intestinal Permeability Enhancers” in “Drug Absorption Enhancement” (ed.) Boer, Harwood Academic Publishers. Such absorption enhancers include, for example, fatty acids, monoglycerides, and bile acids, e.g., as described supra.

Because the compositions of the present invention provide a clear aqueous dispersion upon mixing with an aqueous medium, they have the advantage of sufficiently solubilizing and effectively presenting any enhancer at the absorption site of the therapeutic agent. For example, chenodeoxycholic acid (CDCA) and ursodeoxycholic acid (UDCA) are known enhancers for promoting the oral absorption of macromolecules. CDCA and UDCA, particularly UDCA, is practically insoluble in water having a pH at about 7 and below. As a result, there is a high probability that these enhancers will exist in their insoluble forms in the stomach and duodenum, thereby limiting their absorption-enhancing activity in a conventional composition. The compositions of the present invention, however, are advantageous in that the absorption enhancer remains solubilized in the aqueous environment of the stomach and/or intestines following oral administration of the composition.

As another example, glycerol monooleate, like other hydrophobic enhancers, is practically water insoluble. In the absence of sufficient dispersion and/or solubilization, glycerol monooleate compositions form a turbid and coarse emulsion of large oil droplets that have little absorption enhancement activity. However, the combination of triglyceride and surfactants of the present invention enables the solubilization of glycerol mono-oleate in a clear aqueous dispersion, thereby facilitating the absorption-enhancing ability of glycerol monooleate.

Faster dissolution and release: Due to the robustness of compositions of the present invention to dilution, the therapeutic agents remain solubilized and thus do not suffer problems of precipitation of the therapeutic agent in the time frame relevant for absorption. In addition, the therapeutic agent is presented in small particle carriers, and is not limited in dilution rate by entrapment in emulsion carriers. These factors avoid liabilities associated with the poor partitioning of lipid solubilized drug in to the aqueous phase, such as large emulsion droplet surface area, and high interfacial transfer resistance, and enable rapid completion of the critical partitioning step.

Consistent performance: Aqueous dispersions of the present invention are thermodynamically stable for the time period relevant for absorption, and can be more predictably reproduced, thereby limiting variability in bioavailability—a particularly important advantage for therapeutic agents with a narrow therapeutic index.

Efficient release: The compositions of the present invention are designed with components that help to keep the therapeutic agent solubilized for transport to the absorption site and readily available for absorption, thus providing a more efficient transport and release.

Less likelihood of gastric emptying delays: Unlike conventional triglyceride-containing compositions, the present compositions are less prone to gastric emptying delays, resulting in faster absorption. Further, the particles in dispersions of the present invention are less prone to unwanted retention in the gastro-intestinal tract.

Improved delivery of the therapeutic agent: As discussed previously, the delivery of the therapeutic agent is improved with respect to the extent, rate, and/or consistency of the absorption of the therapeutic agent. The improved delivery is a result of improved loading and solubilization of the triglyceride, the surfactant, and/or the therapeutic agent in the present compositions and in the aqueous dispersions thereof, as indicated, for example, by the clarity of the aqueous dispersion. In one approach, the delivery of the therapeutic agent is enhanced as a result of an increased amount of the therapeutic agent in a readily absorbable form. Delivery of hydrophobic therapeutic agents, such as fenofibrate, progesterone, and cyclosporin, may be enhanced based on this approach.

These and other advantages of the present invention, as well as aspects of preferred embodiments, are illustrated more fully in the Examples that follow.

### EXAMPLE 1

### Preparation of Compositions

A simple pre-concentrate is prepared as follows. Predetermined weighed amounts of the surfactants and triglyceride are stirred together to form a homogeneous mixture. For combinations that are poorly miscible, the mixture can be gently heated to aid in formation of the homogeneous mixture. If the composition is to include a therapeutic agent, the chosen therapeutic agent in a predetermined amount is added and stirred until solubilized. Optionally, solubilizers or additives are included by simple mixing.

To form an aqueous dispersion of the pre-concentrate, a predetermined amount of purified water, buffer solution, or aqueous simulated physiological solution, is added to the pre-concentrate, and the resultant mixture is stirred to form a clear, aqueous dispersion.

### EXAMPLE 2

### Triglyceride Solubilization in Conventional Compositions

Conventional compositions of a triglyceride and a hydrophilic surfactant were prepared for comparison to compositions of the present invention. For each surfactant-triglyceride pair, multiple dispersions were prepared with differing amounts of the two components, to determine the maximum amount of the triglyceride that can be present while the composition still forms a clear dispersion upon a 100-fold dilution with distilled water. No therapeutic agent was included in these compositions, since it is believed that the presence of the therapeutic agent does not substantially affect the clear, aqueous nature of the composition. For the same reason, these compositions were free of additional solubilizers and other additives. The optical clarity was determined by visual inspection and/or by UV absorption (at 400 nm). When UV absorption was used, compositions were considered to be clear when the absorption was less than about 0.2.

Table 20 shows the maximum amount of triglyceride present in such binary mixtures forming clear aqueous dispersions. The numerical entries in the Table are in units of grams of triglyceride per 100 grams of hydrophilic surfactant.

TABLE 20
BINARY TRIGLYCERIDE-SURFACTANT SOLUBILITY

| | Hydrophilic Surfactant | | | | | | |
|---|---|---|---|---|---|---|---|
| | PEG-35 | PEG-40H | PEG-6 | PEG-60 | PEG-45 Palm | | |
| | Castor Oil | Castor Oil | Caprate/ | Corn Oil | Kernel Oil | Polysorbate- | Polysorbate- |
| | (Incrocas | (Cremophor | Caprylate | (Crovol PK- | (Crovol PK- | 20 | 80 |
| Triglyceride | 35) | RH-40) | (Softigen | 70) | 70) | (Tween 20) | (Tween 80) |
| CornOil | 10 | 25 | 3 | 5 | 8 | 2 | 10 |
| (Croda, Super | | | | | | | |
| Refined | | | | | | | |
| Soybean Oil | 10 | 25 | 3 | 8 | 8 | 2 | 10 |
| (Croda, Super | | | | | | | |
| Refined | | | | | | | |
| Glyceryl | 60 | 40 | 8 | 30 | 25 | 20 | 45 |
| Tricaprylate/ | | | | | | | |
| Caprate (Captex | | | | | | | |
| 300) | | | | | | | |
| Glyceryl | 70 | 40 | 5 | 55 | 30 | 20 | 55 |
| Tricaprylate/ | | | | | | | |
| Caprate (Captex | | | | | | | |
| 355 | | | | | | | |
| Glyceryl | 70 | 60 | 5 | 55 | 25 | 10 | 50 |
| Tricaprylate/ | | | | | | | |
| Caprate/Laurate | | | | | | | |
| (Captex 350) | | | | | | | |
| Glyceryl | 30 | 40 | 3 | 25 | 15 | 2 | 25 |
| Tricaprylate/ | | | | | | | |
| Caprate/Linoleate | | | | | | | |
| (Captex 810D) | | | | | | | |

### EXAMPLE 3

### Effect of Surfactant Combinations

The procedure of Example 2 was repeated for compositions containing PEG-40 hydrogenated castor oil (Cremophor RH 40) or polysorbate 80 (Tween 80) as the hydrophilic surfactant, but substituting a second hydrophilic surfactant (compositions number 6-7 and 14-16) or a hydrophobic surfactant (compositions number 4-5,8-9, and 17-18) for part of the hydrophilic surfactant. The total amount of hydrophilic surfactant was kept constant. The results are summarized in Table 21.

TABLE 21A
EFFECTS OF SURFACTANT COMBINATIONS ON THE
SOLUBILIZATION OF TRIGLYCERIDES
Composition in w/w ratio

| | 1 | 2 | 3 | 4 | 5 | 6 | 7 | 8 | 9 |
|---|---|---|---|---|---|---|---|---|---|
| Corn | 25 | 30 | 40 | 40 | 40 | 40 | 40 | 40 | 40 |
| Oil | | | | | | | | | |
| Cremo- | 100 | 100 | 100 | 77 | 71 | 67 | 57 | 62 | 57 |
| phor | | | | | | | | | |
| RH-40 | | | | | | | | | |
| Peceol | — | — | — | 23 | 29 | — | — | — | — |
| Kessco | — | — | — | — | — | 33 | 43 | — | — |
| PEG | | | | | | | | | |
| 400 MO | | | | | | | | | |
| Crovol | — | — | — | — | — | — | — | 38 | 43 |
| M-40 | | | | | | | | | |
| Appear- | Clear | Hazy | Hazy | Clear | Clear | Clear | Clear | Hazy | Hazy |
| ance | | | | | | | | | |
| of the | | | | | | | | | |
| Concen- | | | | | | | | | |
| trate | | | | | | | | | |
| Abs @ | 0.148 | 2.195 | 2.518 | 0.121 | 0.132 | 0.124 | 0.102 | 0.233 | 0.167 |
| 400 nm | | | | | | | | | |
| of the | | | | | | | | | |
| 100X | | | | | | | | | |
| (w/v) | | | | | | | | | |
| Dilution | | | | | | | | | |
| in | | | | | | | | | |
| Deion- | | | | | | | | | |
| ized | | | | | | | | | |
| Water | | | | | | | | | |

TABLE 21B
Composition in w/w ratio

| | 10 | 11 | 12 | 13 | 14 | 15 | 16 | 17 | 18 |
|---|---|---|---|---|---|---|---|---|---|
| Corn | 10 | 15 | 20 | 30 | 15 | 20 | 30 | 20 | 25 |
| Oil | | | | | | | | | |
| Tween | 100 | 100 | 100 | 100 | 67 | 67 | 67 | 67 | 67 |
| 80 | | | | | | | | | |
| Kessco | — | — | — | — | 33 | 33 | 33 | — | — |
| PEG | | | | | | | | | |
| 400 | | | | | | | | | |
| MO | | | | | | | | | |
| Crovol | — | — | — | — | — | — | — | 33 | 33 |
| M-40 | | | | | | | | | |
| Appear- | Clear | Clear | Hazy | Hazy | Clear | Clear | Clear | Clear | Clear |
| ance | | | | | | | | | |
| of the | | | | | | | | | |
| Concen- | | | | | | | | | |
| trate | | | | | | | | | |
| Abs | 0.002 | 1.314 | 1.613 | 1.654 | 0.041 | 0.019 | 0.194 | 0.057 | 0.158 |
| @ | | | | | | | | | |
| 400 nm | | | | | | | | | |
| of the | | | | | | | | | |
| 100X | | | | | | | | | |
| (w/v) | | | | | | | | | |
| Dilution | | | | | | | | | |
| in | | | | | | | | | |
| Deion- | | | | | | | | | |
| ized | | | | | | | | | |
| Water | | | | | | | | | |

The clear or hazy appearance noted in the Table is that of the pre-concentrate, not of the aqueous dispersion. The clarity of the aqueous dispersion is shown quantitatively by UV absorption of the 100× dilution at 400 nm.

Comparing compositions 1-3, a binary corn oil-Cremophor RH-40 mixture having 25 grams of corn oil per 100 grams of the surfactant is optically clear, having an absorption of 0.148. However, upon a slight increase of the amount of corn oil to 30 grams, the dispersion becomes cloudy, with an absorbance of 2.195, indicating the formation of a conventional emulsion. Compositions 4-5 show the surprising result that when part of the hydrophilic Cremophor RH-40 is replaced by a hydrophobic surfactant (Peceol), keeping the total surfactant concentration constant, compositions having a much higher amount of triglyceride (40 grams) still form clear aqueous dispersions, with absorbances less than 0.2 and dramatically less than the comparable binary composition number 3. A similar result is shown in compositions 8-9 for a different hydrophobic surfactant, Crovol M-40. Likewise, when part of the hydrophilic surfactant is replaced by a second hydrophilic surfactant in compositions 6-7, it is surprisingly found that the amount of triglyceride solubilized is similarly increased.

The second part of the Table, Table 21B, shows a similar surprising result for a different hydrophilic surfactant, Tween 80. Simple binary corn oil-Tween 80 mixtures form clear aqueous dispersions with 10 grams of corn oil, but are cloudy and multi-phasic with 15 grams or more of the triglyceride. As the Table shows, substitution of part of the hydrophilic surfactant with a second hydrophilic surfactant or a hydrophobic surfactant dramatically increases the amount of triglyceride that can be solubilized.

### EXAMPLE 4

### Effect of Surfactant Combinations

Example 3 was repeated, using different triglyceride-surfactant combinations. In particular, medium-chain triglycerides (MCTs) were used instead of corn oil, a long-chain triglyceride (LCT). The results are shown in the three-part Table 22.

TABLE 22A
SOLUBILIZATION OF MCTs
Composition in w/w ratio

| | 19 | 20 | 21 | 22 | 23 |
|---|---|---|---|---|---|
| Pureco 76 | 33 | 50 | 80 | 50 | 80 |
| Cremophor RH-40 | 100 | 100 | 100 | 40 | 100 |
| Imwitor 988 | — | — | — | 60 | 100 |
| Ethanol | — | — | — | — | 33 |
| Appearance of the | Clear | Clear | Hazy | Clear | Clear |
| Concentrate | | | | | |
| Abs @ 400 nm of the | 0.201 | 0.346 | 2.522 | 0.204 | 0.098 |
| 100X (w/v) Dilution in | | | | | |
| Deionized Water | | | | | |

TABLE 22B
Composition in w/w ratio

| | 24 | 25 | 26 | 27 |
|---|---|---|---|---|
| Captex 300 | 40 | 75 | 75 | 75 |
| Cremophor RH-40 | 100 | 100 | 50 | 100 |
| Imwitor 988 | — | — | 50 | 75 |
| Appearance of the | Clear | Hazy | Clear | Clear |
| Concentrate | | | | |
| Abs @ 400 nm of the | 0.180 | 0.557 | 0.208 | 0.078 |
| 100X (w/v) Dilution in | | | | |
| Deionized Water | | | | |

TABLE 22C
Composition in w/w ratio

| | 28 | 29 | 30 | 31 | 32 | 33 |
|---|---|---|---|---|---|---|
| Captex 300 | 20 | 25 | 33 | 30 | 40 | 40 |
| Tween 20 | 100 | 100 | 100 | 70 | 70 | 66 |
| Brij 30 | — | — | — | 30 | 30 | 34 |
| Appearance | Clear | Hazy | Hazy | Clear | Clear | Clear |
| of the | | | | | | |
| Concentrate | | | | | | |
| Abs @ | 0.078 | 1.192 | 2.536 | 0.017 | 0.234 | 0.103 |
| 400 nm of the | | | | | | |
| 100X (w/v) | | | | | | |
| Dilution in | | | | | | |
| Deionized | | | | | | |
| Water | | | | | | |

Table 22 shows that the increased solubilization of the triglyceride is observed for MCTs as well as for LCTs, with a variety of surfactants. Table 22 additionally shows that the same effect is observed in the presence of increased amounts of surfactants (compositions 23 and 27) and solubilizers (composition 23).

### EXAMPLE 5

### Characterization of Compositions

Various compositions were prepared and characterized by visual observation as well as by UV absorbance at 400 nm. Each composition was diluted 100-fold with distilled water. The results are shown in Table 23.

TABLE 23
VISUAL AND SPECTROSCOPIC CHARACTERIZATION

| | | | | Absorbance |
|---|---|---|---|---|
| No. | Composition | | Visual Observation | at 400 nm |
| 24 | Soybean Oil | 80 mg | Very clear solution | 0.014 |
| | Tween 20 | 200 mg | | |
| | Tween 80 | 800 mg | | |
| 25 | Captex 810D | 250 mg | Very clear solution | 0.030 |
| | Incrocas 35 | 500 mg | | |
| | Tween 80 | 500 mg | | |
| 26 | Captex 810D | 200 mg | Clear solution | 0.157 |
| | Incrocas 35 | 667 mg | | |
| | Myvacet 9-45 | 333 mg | | |
| 27 | Corn Oil | 250 mg | Clear solution | 0.085 |
| | Cremophor RH-40 | 750 mg | | |
| | Peceol | 150 mg | | |
| | Propylene Glycol | 100 mg | | |
| 28 | Captex 355 | 200 mg | Clear solution | 0.212 |
| | Labrafil M2125CS | 300 mg | | |
| | Cremophor RH-40 | 500 mg | | |
| | Ethanol | 100 mg | | |
| 29 | Captex 355 | 150 mg | Clear solution | 0.141 |
| | Cremophor RH-40 | 600 mg | | |
| | Labrafil M2125CS | 250 mg | | |
| | Ethanol | 100 mg | | |
| 30 | Captex 355 | 300 mg | Clear solution, | 0.241 |
| | Incrocas 35 | 500 mg | Slightly hazy | |
| | Labrafil M2125CS | 200 mg | | |
| | Ethanol | 100 mg | | |
| 31 | Captex 355 | 250 mg | Clear solution | 0.076 |
| | Incrocas 35 | 600 mg | | |
| | Labrafil M2125CS | 150 mg | | |
| | Ethanol | 100 mg | | |
| 32 | Pureco 76 | 160 mg | Clear solution | 0.168 |
| | Cremophor RH-40 | 480 mg | | |
| | Labrafil M2125CS | 160 mg | | |
| | Ethanol | 150 mg | | |

### EXAMPLE 6

### COMPARATIVE EXAMPLE

Prior art compositions were prepared for comparison with the compositions of the present invention. As in Example 5, the compositions were diluted 100-fold with distilled water, and characterized by visual observation and by UV absorbance. The results are shown in Table 24.

TABLE 24
COMPOSITIONS NOT FORMING CLEAR AQUEOUS DISPERSIONS

| | | | Visual | Absorbance |
|---|---|---|---|---|
| No. | Composition | | Observation | at 400 nm |
| 33 | Corn Oil | 400 mg | Milky suspension | 1.989 |
| | Cremophor RH-40 | 710 mg | | |
| | Crovol M-40 | 290 mg | | |
| 34 | Captex 300 | 300 mg | Milky suspension | 1.594 |
| | Tween 20 | 650 mg | | |
| | Imwitor 988 | 350 mg | | |
| 35 | Corn Oil | 400 mg | Milky suspension | 2.716 |
| | Cremophor RH-40 | 620 mg | | |
| | Labrafil M2125CS | 380 mg | | |
| 36 | Soybean Oil | 185 mg | Milky suspension | 2.595 |
| | Captex GTO | 275 mg | | |
| | Tween 80 | 275 mg | | |
| | Triacetin | 185 mg | | |
| 37 | Pureco 76 | 315 mg | Milky suspension | 2.912 |
| | Cremophor RH-40 | 225 mg | | |
| | Span 20 | 360 mg | | |
| 38 | Soybean Oil | 340 mg | Milky suspension | 2.566 |
| | Captex GTO | 280 mg | | |
| | Tween 80 | 280 mg | | |
| 39 | Pureco 76 | 330 mg | Milky suspension | 2.233 |
| | Labrasol | 120 mg | | |
| 40 | Corn Oil | 400 mg | Milky suspension | 2.249 |
| | Cremophor RH-40 | 570 mg | | |
| | Lauroglycol FCC | 430 mg | | |
| 41 | Soybean Oil | 160 mg | Milky suspension | 2.867 |
| | Tween 80 | 200 mg | | |
| | Imwitor 988 | 450 mg | | |
| | Ethanol | 150 mg | | |
| 42 | Corn Oil | 200 mg | Milky suspension | 1.547 |
| | Tween 80 | 570 mg | | |
| | Kessco PEG 400 MO | 430 mg | | |

As the Table shows, conventional compositions such as those disclosed in U.S. Pat. No. 5,645,856, form milky suspensions rather than the clear aqueous dispersions of the present invention.

### EXAMPLE 7

### Comparison with Triglyceride-Containing Compositions of WO 99/29300

Three compositions of the invention were prepared with the components indicated in Table 25, then diluted 100-fold with distilled water and characterized by UV absorbance at 400 nm as in the preceding examples. Then, each of the compositions was prepared with 100 mg/g of fenofibrate, and the absorbance of those compositions following 100-fold dilution with distilled water was determined as well. The results are given in Table 25:

TABLE 25

| | Composition 43 | Composition 44 | Composition 45 |
|---|---|---|---|
| Components | parts by wt. | parts by wt. | parts by wt. |
| Capmul MCM | 30 | 30 | 17 |
| Crem RH 40 | 30 | — | — |
| Crem EL | — | 30 | 43 |
| Miglyol 812 N | 28 | 28 | 40 |
| Labrasol | — | — | — |
| Clarity | 0.298 | 0.255 | 0.257 |
| (Absorbance @ | | | |
| 400 nm of | | | |
| 100X), with | | | |
| 100 mg/g feno- | | | |
| fibrate in the | | | |
| composition: | | | |

For purposes of comparison, four compositions exemplified in International Patent Publication No. WO 99/29300 were prepared and evaluated in the same manner. The compositions prepared are those of Examples 1, 2, 6, and 7 of the aforementioned patent publication. The results are set forth in Table 26:

TABLE 26

| | WO | WO | WO | WO |
|---|---|---|---|---|
| Composition | 99/29300, | 99/29300, | 99/29300, | 99/29300, |
| Components | Example 1 | Example 2 | Example 6 | Example 7 |
| Miglyol 810 | 23 | 23 | 26 | 28 |
| Linoleic Acid | 12 | 12 | 12 | 13 |
| Tween-80 | 18 | 35 | 49 | 48 |
| Myrj 52 | 15 | — | — | — |
| TPGS | 10 | 7 | — | — |
| Labrasol | — | — | 2 | 5 |
| Ethanol | 11 | 12 | 11 | 4 |
| Propylene Glycol | 11 | 12 | — | 2 |
| Clarity (Absorbance @ | 0.759 | 0.594 | 0.88 | 2.677 |
| 400 nm of 100X), with | | | | |
| 100 mg/g fenofibrate in | | | | |
| the composition: | | | | |

As may be deduced from the data in Table 25, the compositions of the invention provide for a very clear aqueous dispersion upon 100× dilution with distilled water, even when 100 mg/g of drug (i.e., fenofibrate) is incorporated. By contrast, as Table 26 illustrates, the compositions of WO 99/29300 do not provide for clear dispersions upon 100× dilution when 100 mg/g fenofibrate is incorporated, nor does a corresponding fenofibrate-free composition necessarily result in a clear aqueous dispersion.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope. All patents, patent applications, patent publications and non-patent literature references mentioned herein are incorporated by reference in their entireties.

We claim;

1. A pharmaceutical composition for oral administration of a lipid-regulating agent, comprising:
(a) a carrier comprising a triglyceride and at least two surfactants, at least one of the surfactants being hydrophilic; and
(b) a therapeutically effective amount of a lipid-regulating agent,
wherein the triglyceride and surfactants are present in amounts that are pharmaceutically acceptable and are selected so that upon admixture of the composition with an aqueous medium in an aqueous medium to composition ratio of about 100:1 by weight, a clear aqueous dispersion having an absorbance of less than about 0.3 at 400 nm is provided.

2. The composition of claim 1, wherein the lipid-regulating agent is capable of being solubilized in the triglyceride, the surfactants, or both the triglyceride and the surfactants.

3. The composition of claim 1, wherein the lipid-regulating agent is a fibric acid derivative.

4. The composition of claim 1, wherein the lipid-regulating agent is selected from bezafibrate, beclobrate, binifibrate, ciprofibrate, clinofibrate, clofibrate, etofibrate, fenofibrate, gemfibrozil, nicofibrate, pirifibrate, ronifibrate, simfibrate, and theofibrate.

5. The composition of claim 4, wherein the lipid-regulating agent is fenofibrate.

6 The composition of claim 1, wherein the lipid-regulating agent is a sterol absorption inhibitor.

7. The composition of claim 6, wherein the sterol absorption inhibitor is ezemitibe.

8. The composition of claim 1, wherein the lipid-regulating agent is an HMG CoA reductase inhibitor.

9. The composition of claim 8, wherein the lipid-regulating agent is selected from atorvastatin, cerivastatin, fluindostatin, fluvastatin, lovastatin, mevastatin, nystatin, pitivastatin, pravastatin, rosuvastatin, simvastatin, urinastatin, and velostatin.

10. The composition of claim 8, wherein the lipid-regulating agent is selected from atorvastatin, simvastatin, pravastatin, and lovastatin.

11. The composition of claim 1, wherein the lipid-regulating agent is selected from nicotinic acid, nicomol, and niceritrol.

12. The composition of claim 1, wherein the lipid-regulating agent is a bile acid sequestrant, probucol, a squalene synthesis inhibitor, or an LDL catabolism enhancer.

13. The composition of claim 1, further comprising a second therapeutic agent.

14. The composition of claim 13, wherein said second therapeutic agent is selected from the group consisting of an additional lipid-regulating agent, an anti-diabetic agent, an anti-coagulant, an antihypertensive agent, and mixtures thereof.

15. The composition of claim 14, wherein the additional lipid-regulating agent is selected from the group consisting of atorvastatin, cerivastatin, fluindostatin, fluvastatin, lovastatin, mevastatin, nystatin, pitivastatin, pravastatin, rosuvastatin, simvastatin, urinastatin, velostatin, nicotinic acid, nicomol, niceritrol, and mixtures thereof.

16. The composition of claim 14, wherein the anti-diabetic agent is selected from the group consisting of sulfonyl ureas, biguanides, thiazolidinediones, alpha glucosidase inhibitors, and meglitinides.

17. The composition of claim 14, wherein the anti-diabetic agent is selected from the group consisting of glyburide, metformin, glipizide, glimepiride, pioglitazone, rosiglitazone, miglitol, acarbose, repaglinide, nateglinide, and mixtures thereof.

18. The composition of claim 1, wherein said triglyceride is a medium chain triglyceride.

19. The composition of claim 18, wherein said medium chain triglyceride is glyceryl tricaprylate/caprate.

20. The composition of claim 18, wherein said medium chain triglyceride is a triglyceride composition in which at least 50% of the fatty acids have a chain length of 6 to 12 carbon atoms.

21. The composition of claim 1, wherein the at least one hydrophilic surfactant is selected from the group consisting of polyoxyethylene sorbitan fatty acid esters, polyoxyethylene vegetable oil, polyoxyethylene hydrogenated vegetable oil and mixtures thereof.

22. The composition of claim 21, wherein the at least one hydrophilic surfactant is selected from polysorbate 80, PEG-35 castor oil, and PEG-40 castor oil.

23. The composition of claim 1, wherein the at least one hydrophilic surfactant is selected from the group consisting of hydrophilic transesterification products of oils and alcohols and mixtures thereof.

24. The composition of claim 23, wherein the oil is an oil-soluble vitamin

25. The composition of claim 23, wherein the at least one hydrophilic surfactant is tocopheryl PEG-1000 succinate.

26. The composition of claim 23, wherein the at least one hydrophilic surfactant is selected from the group consisting of PEG-8 caprylic/capric glycerides, lauroyl macrogol-32 glycerides, stearoyl macrogol glyceride, and mixtures thereof.

27. The composition of claim 1, wherein the carrier further comprises at least one hydrophobic surfactant.

28. The composition of claim 27, wherein the at least one hydrophobic surfactant is selected from hydrophobic transesterification products of oils and alcohols and mixtures thereof.

29. The composition of claim 28, wherein the at least one hydrophobic surfactant is selected from transesterification products of PEG-6 corn oil, PEG-6 apricot kernel oil, and mixtures thereof.

30. The composition of claim 27, wherein the at least one hydrophobic surfactant is a glycerol fatty acid ester selected from monoglycerides, diglycerides, and mixtures thereof.

31. The composition of claim 30, wherein the at least one hydrophobic surfactant is a glycerol fatty acid ester selected from glyceryl caprylate, glyceryl caprylate/caprate and mixtures thereof.

32. The composition of claim 30, wherein the at least one hydrophobic surfactant is selected from glyceryl linoleate, glyceryl monooleate, glyceryl dioleate and mixtures thereof.

33. The composition of claim 27, wherein the at least one hydrophobic surfactant is a propylene glycol fatty acid ester.

34. The composition of claim 33, wherein the propylene glycol fatty acid ester is selected from propylene glycol monocaprylate, propylene glycol dicaprylate/dicaprate, and mixtures thereof.

35. The composition of claim 1, further comprising a solubilizer.

36. The composition of claim 2, wherein the lipid-regulating agent is solubilized in the triglyceride, the surfactants, or both the triglyceride and the surfactants.

37. The composition of claim 36, further comprising an additional amount of the lipid-regulating agent that is not solubilized.

38. The composition of claim 1, further comprising an antioxidant.

39. The composition of claim 1, further comprising a viscomodulator.

40. The composition of claim 1, further comprising a suspending agent.

41. A dosage form comprising the composition of claim 1, wherein said dosage form is a capsule.

42. The dosage form of claim 41, wherein the capsule is a starch, hydroxypropyl methylcellulose, or gelatin capsule.

43. The dosage form of claim 42, wherein the capsule is a hard gelatin capsule or a soft gelatin capsule.

44. The composition of claim 1, wherein the absorbance of the clear aqueous dispersion formed is less than about 0.2 at 400 nm.

45. The composition of claim 44, wherein the absorbance of the clear aqueous dispersion formed is less than about 0.1 at 400 nm.

46. The composition of claim 1, comprising a preconcentrate in a liquid, semi-solid, or solid form, or an aqueous or organic diluted preconcentrate.

47. The composition of claim 1, wherein the composition is substantially free of water.

48. The composition of claim 1, wherein the triglyceride and surfactants are present in amounts such that upon admixture of the composition with an aqueous medium in an aqueous medium to composition ratio of about 10:1 by weight, a clear aqueous dispersion having an absorbance of less than about 0.3 at 400 nm is provided.

49. The composition of claim 48, wherein the absorbance of the clear aqueous dispersion is less than about 0.2 at 400 nm.

50. The composition of claim 49, wherein the absorbance of the clear aqueous dispersion is less than about 0.1 at 400 nm.

51. The composition of claim 1, wherein the therapeutically effective amount is in a unit dosage.

52. The composition of claim 1, wherein following oral administration, the composition provides an increase in the rate of absorption of the lipid-regulating agent relative to the rate of absorption for a corresponding composition, administered under an identical dosage regimen, containing (a) the lipid-regulating agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization.

53. The composition of claim 52, wherein the increase in the rate of absorption is such that the time to reach maximum plasma concentration of the lipid-regulating agent or an active metabolite thereof is reduced by at least about 10%.

54. The composition of claim 53, wherein the time to reach maximum plasma concentration of the lipid-regulating agent or an active metabolite thereof is reduced by at least about 20%.

55. The composition of claim 54, wherein the time to reach maximum plasma concentration of the lipid-regulating agent or an active metabolite thereof is reduced by at least about 50%.

56. The composition of claim 5, wherein following oral administration, the composition provides an increase in the rate of absorption of fenofibrate relative to the rate of absorption for a corresponding composition, administered under an identical dosage regimen, containing (a) fenofibrate, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization.

57. The composition of claim 56, wherein the increase in the rate of absorption is such that the time to reach maximum plasma concentration of fenofibric acid is reduced by at least 10%.

58. The composition of claim 57, wherein the increase in the rate of absorption is such that the time to reach maximum plasma concentration of fenofibric acid is reduced by at least 20%.

59. The composition of claim 58, wherein the increase in the rate of absorption is such that the time to reach maximum plasma concentration of fenofibric acid is reduced by at least 50%.

60. The composition of claim 1, wherein following oral administration, the composition provides an increase in the extent of absorption of the lipid-regulating agent relative to the extent of absorption for a corresponding composition, administered under an identical dosage regimen, containing (a) the lipid-regulating agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization.

61. The composition of claim 60, wherein the increase in the extent of absorption is such that the area under the curve (AUC) of the plasma concentration of the lipid-regulating agent or an active metabolite thereof is increased by at least about 10%.

62. The composition of claim 61, wherein the increase in the extent of absorption is such that the area under the curve (AUC) of the plasma concentration of the lipid-regulating agent or an active metabolite thereof is increased by at least about 20%.

63. The composition of claim 62, wherein the increase in the extent of absorption is such that the area under the curve (AUC) of the plasma concentration of the lipid-regulating agent or an active metabolite thereof is increased by at least about 50%.

64. The composition of claim 5, wherein following oral administration, after dose normalization, the composition provides an increase in the extent of absorption of fenofibrate relative to the extent of absorption for a corresponding composition, administered under an identical dosage regimen, containing (a) fenofibrate, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight.

65. The composition of claim 64, wherein the increase in the extent of absorption is such that the area under the curve (AUC) of the plasma concentration of fenofibric acid is increased by at least about 10%.

66. The composition of claim 65, wherein the increase in the extent of absorption is such that the area under the curve (AUC) of the plasma concentration of fenofibric acid is increased by at least about 20%.

67. The composition of claim 66, wherein the increase in the rate of absorption is such that the time to reach maximum plasma concentration of fenofibric acid is reduced by at least 50%.

68. The composition of claim 5, wherein the composition provides an AUC_{0-inf} of the plasma concentration of fenofibric acid, per milligram of dosed fenofibrate, of at least 1.25 μg·hr/ml, following oral administration of the composition to a human patient.

69. A pharmaceutical composition comprising:
(a) a carrier comprising a triglyceride and at least two surfactants, at least one of the surfactants being hydrophilic; and
(b) a therapeutically effective amount of a lipid-regulating agent,
wherein the triglyceride and the surfactants are present in amounts that are pharmaceutically acceptable and selected so that upon admixture of the composition with an aqueous medium in an aqueous medium to composition ratio of about 10:1 by weight, a clear aqueous dispersion is provided.

70. A pharmaceutical composition for oral administration, comprising:
(a) a carrier comprising a medium chain triglyceride and at least two surfactants, at least one of the surfactants being a hydrophilic surfactant selected from polyoxyethylene sorbitan fatty acid esters, polyoxyethylene vegetable oil, polyoxyethylene hydrogenated vegetable oil, and hydrophilic transesterification products of oils and alcohol and mixtures thereof; and
(b) a therapeutically effective amount of a lipid-regulating agent selected from bezafibrate, beclobrate, binifibrate, ciprofibrate, clinofibrate, clofibrate, etofibrate, fenofibrate, gemfibrozil, nicofibrate, pirifibrate, ronifibrate, simfibrate, theofibrate, and the acid forms thereof,
wherein the triglyceride and surfactants are present in amounts that are pharmaceutically acceptable and are selected so that upon admixture of the composition with an aqueous medium in an aqueous medium to composition ratio of about 100:1 by weight, a clear aqueous dispersion having an absorbance of less than about 0.3 at 400 nm is provided.

71. The composition of claim 70, wherein the carrier contains a hydrophilic surfactant.

72. The composition of claim 71, wherein the hydrophilic surfactant is a hydrophilic transesterification product of an oil and an alcohol.

73. The composition of claim 72, wherein the oil is an oil-soluble vitamin.

74. The composition of claim 70, wherein the carrier further includes at least one hydrophobic surfactant selected from (a) hydrophobic transesterification products of an oil and an alcohol; and (b) glycerol fatty acid esters selected from monoglycerides, diglycerides, and mixtures thereof.

75. The composition of claim 70, wherein the lipid-regulating agent is fenofibrate and the therapeutically effective amount is a unit dosage.

76. A pharmaceutical composition comprising:
(a) a carrier comprising glyceryl tricaprylate/caprate, at least one hydrophilic surfactant selected from the group consisting of tocopheryl PEG-1000 succinate, polysorbate 80, PEG-35 castor oil, PEG-40 hydrogenated castor oil, PEG-8 caprylic/capric glycerides, lauroyl macrogol-32 glycerides, stearoyl macrogol glyceride, and mixtures thereof; and at least one hydrophobic surfactant selected from the group consisting of glyceryl caprylate, glyceryl caprylate/caprate and mixtures thereof; and
(b) a therapeutically effective amount of fenofibrate,
wherein the triglyceride and surfactants are present in amounts that are pharmaceutically acceptable and selected so that upon admixture of the composition with an aqueous solution in an aqueous solution to composition ratio of about 100:1 by weight, a clear aqueous dispersion having an absorbance of less than about 0.3 at 400 nm is provided.

77. The composition of claim 76, wherein the at least one hydrophilic surfactant is selected from the group consisting of tocopheryl PEG-1000 succinate, polysorbate 80, and mixtures thereof.

78. The composition of claim 76, wherein following oral administration, the composition provides an increase in the rate of absorption of fenofibrate relative to the rate of absorption for a corresponding composition in the form of a hard gelatin capsule containing fenofibrate in a dry powder form, wherein said fenofibrate is co-micronized with sodium lauryl sulfate, after dose normalization.

79. The composition of claim 78, wherein the increase in the rate of absorption is such that the time to reach maximum plasma concentration of fenofibric acid is reduced by at least 25%.

80. The composition of claim 79, wherein the increase in the rate of absorption is such that the time to reach maximum plasma concentration of fenofibric acid is reduced by at least 50%.

81. The composition of claim 80, wherein the increase in the rate of absorption is such that the time to reach maximum plasma concentration of fenofibric acid is reduced by at least 75%.

82. The composition of claim 76, wherein following oral administration, the composition provides an increase in the extent of absorption of the lipid-regulating agent relative to the extent of absorption for a corresponding composition in the form in the form of a hard gelatin capsule containing fenofibrate in a dry powder form, wherein said fenofibrate is co-micronized with sodium lauryl sulfate, after dose normalization.

83. The composition of claim 82, wherein the increase in the extent of absorption is such that the area under the curve (AUC) of the plasma concentration of fenofibric acid is increased by at least about 25%.

84. The composition of claim 83, wherein the increase in the extent of absorption is such that the area under the curve (AUC) of the plasma concentration of fenofibric acid thereof is increased by at least about 50%.

85. The composition of claim 84, wherein the increase in the extent of absorption is such that the area under the curve (AUC) of the plasma concentration of fenofibric acid is increased by at least about 75%.

86. A pharmaceutical composition for oral administration of a lipid-regulating agent, comprising:
(a) a carrier comprising a triglyceride and at least two surfactants, at least one of the surfactants being hydrophilic; and
(b) a therapeutically effective amount of a lipid-regulating agent selected from bezafibrate, beclobrate, binifibrate, ciprofibrate, clinofibrate, clofibrate, etofibrate, fenofibrate, gemfibrozil, nicofibrate, pirifibrate, ronifibrate, simfibrate, and theofibrate,
wherein the triglyceride and surfactants are present in amounts selected so that upon admixture of the composition with an aqueous medium in an aqueous medium to composition ratio of about 100:1 by weight, a clear aqueous dispersion is provided, and further wherein following oral administration, the composition provides for absorption of the lipid-regulating agent or an active metabolite thereof that is less dependent upon lipolysis relative to a corresponding composition, administered under an identical dosage regimen, containing (a) the lipid-regulating agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization.

87. The composition of claim 86, wherein the lipid-regulating agent is fenofibrate.

88. A pharmaceutical composition for oral administration of a lipid-regulating agent, comprising:
(a) a carrier comprising a triglyceride and at least two surfactants, at least one of the surfactants being hydrophilic; and
(b) a therapeutically effective amount of a lipid-regulating agent is selected from bezafibrate, beclobrate, binifibrate, ciprofibrate, clinofibrate, clofibrate, etofibrate, fenofibrate, gemfibrozil, nicofibrate, pirifibrate, ronifibrate, simfibrate, and theofibrate,
wherein the triglyceride and surfactants are present in amounts selected so that upon admixture of the composition with an aqueous medium in an aqueous medium to composition ratio of about 100:1 by weight, a clear aqueous dispersion is provided, and further wherein following oral administration, the composition provides for absorption of the lipid-regulating agent or an active metabolite thereof that is less dependent upon endogenous bile, bile-related patient disease states, or meal fat contents lipolysis relative to a corresponding composition, administered under an identical dosage regimen, containing (a) the lipid-regulating agent, (b) at least one hydrophilic surfactant, and (c) at least one lipophilic component selected from a triglyceride, a hydrophobic surfactant, and mixtures thereof, but which results in an aqueous dispersion having an absorbance of greater than 0.5 at 400 nm upon admixture with an aqueous medium in an aqueous medium to composition ratio of about 100:1 by weight, after dose normalization.

89. The composition of claim 88, wherein the lipid-regulating agent is fenofibrate.

90. A method of treating a lipid disorder, comprising administering the composition of claim 1 to a patient in need of such treatment.

91. The method of claim 90, wherein the patient is diabetic.

92. A method of treating a lipid disorder, comprising administering the composition of claim 75 to a patient in need of such treatment.

93. A method of treating a lipid disorder, comprising orally administering to a patient in need of such treatment:
(a) a dosage form of a carrier comprising (i) a triglyceride and (ii) at least two surfactants, at least one of the surfactants being hydrophilic, wherein the triglyceride and surfactants are present in amounts that are pharmaceutically acceptable and selected so that upon mixing with an aqueous solution in an aqueous solution to carrier ratio of about 100:1 by weight, the carrier forms a clear aqueous dispersion having an absorbance of less than about 0.3 at 400 nm; and
(b) a therapeutically effective unit dosage of a lipid-regulating agent.

94. The method of any one of claims 90, 91, 92, or 93, wherein the lipid disorder is hypercholesterolemia, hypertriglyceridemia, or mixed dyslipidemia.

95. The method of any one of claims 90, 91, 92, or 93, wherein the lipid disorder is a metabolic disorder.

96. The method of any one of claims 90, 91, 92, or 93, wherein the lipid disorder is associated with a metabolic syndrome.

97. A method for reducing the dependency of absorption on lipolysis of an orally administered lipid-regulating agent, comprising administering the lipid-regulating agent in a composition that comprises the composition of claim 1.

98. A method for reducing the dependency of absorption on endogenous bile, bile-related patient disease states, or meal fat contents of an orally administered lipid-regulating agent, comprising administering the lipid-regulating agent in a composition that comprises the composition of claim 1.

99. A method of treating a lipid disorder, comprising administering the composition of claim 5 to a patient in need of such treatment.

100. The method of claim 99, wherein the patient is diabetic.

101. A method of treating a lipid disorder, comprising administering the composition of claim 5 to a patient in need of such treatment.

102. A method of treating a lipid disorder, comprising orally administering to a patient in need of such treatment:
(a) a dosage form of a carrier comprising (i) a triglyceride and (ii) at least two surfactants, at least one of the surfactants being hydrophilic, wherein the triglyceride and surfactants are present in amounts that are pharmaceutically acceptable and selected so that upon mixing with an aqueous solution in an aqueous solution to carrier ratio of about 100:1 by weight, the carrier forms a clear aqueous dispersion having an absorbance of less than about 0.3 at 400 nm; and
(b) a therapeutically effective unit dosage of fenofibrate.

103. The method of any one of claims 99, 100, 101, or 102, wherein the lipid disorder is hypercholesterolemia, hypertriglyceridemia, or mixed dyslipidemia.

104. The method of any one of claims 99, 100, 101, or 102, wherein the lipid disorder is a metabolic disorder.

105. The method of claim 104, wherein the lipid disorder is associated with a metabolic syndrome.

106. A method for reducing the dependency of absorption on lipolysis of orally administered fenofibrate, comprising administering the fenofibrate in a composition that comprises the composition of claim 5.

107. A method for reducing the dependency of absorption on endogenous bile, bile-related patient disease states, or meal fat contents of orally administered fenofibrate, comprising administering the fenofibrate in a composition that comprises the composition of claim 5.

108. A method for reducing the dependency of absorption on lipolysis of orally administered fenofibrate, comprising administering the fenofibrate in a composition that comprises the composition of claim 5.

109. A method for reducing the dependency of absorption on endogenous bile, bile-related patient disease states, or meal fat contents of orally administered fenofibrate, comprising administering the fenofibrate in a composition that comprises the composition of claim 5.

110. A pharmaceutical composition for oral administration, comprising:
(a) a carrier comprising a medium chain triglyceride and at least two surfactants, at least one of the surfactants being a hydrophilic surfactant selected from polyoxyethylene sorbitan fatty acid esters, polyoxyethylene vegetable oil, polyoxyethylene hydrogenated vegetable oil, and hydrophilic transesterification products of oils and alcohol and mixtures thereof; and
(b) a therapeutically effective amount of a therapeutic agent,
wherein the triglyceride and surfactants are present in amounts that are pharmaceutically acceptable and are selected so that upon admixture of the composition with an aqueous medium in an aqueous medium to composition ratio of about 100:1 by weight, a clear aqueous dispersion having an absorbance of less than about 0.3 at 400 nm is provided.
